(19) United States
(12) Patent Application Publication
(10) Pub. No.: US 2023/0285520 A1
(43) Pub. Date: Sep. 14, 2023

(54) DEVELOPMENT OF A NEW ENGINEERED TOBACCO ETCH VIRUS (TEV) PROTEASE ACTIVABLE IN THE CYTOSOL OR SECRETORY PATHWAY

(71) Applicants: Università Cattolica del Sacro Cuore, Milan (IT); Fondazione Policlinico Universitario Agostino Gemelli IRCCS, Rome (IT)

(72) Inventors: Claudio GRASSI, Rome (IT); Cristian RIPOLI, Rome (IT); Pietro RENNA, Rome (IT)

(73) Assignees: Università Cattolica del Sacro Cuore, Milano (IT); Fondazione Policlinico Universitario Agostino Gemelli IRCCS, Rome (IT)

(21) Appl. No.: 18/007,300

(22) PCT Filed: Jul. 27, 2021
(86) PCT No.: PCT/IB2021/056788

§ 371 Date: Jan. 27, 2023

(30) Foreign Application Priority Data
Jul. 27, 2020 (IT) 102020000018064

(51) Int. Cl. A61K 38/48; C12N 9/50; C12N 15/10

(52) U.S. Cl. CPC A61K 38/4873; C12N 9/506; C12Y 304/22044; C12N 15/1031; C07K 2319/50

(57) ABSTRACT

The present invention relates to a protein having proteolytic activity inducible and activable by the experimenter in the cytosol or in the secretory pathway, and uses thereof for controlling the maturation in a vital cell of a protein subject to proteolytic cleavage, and in a purification process of recombinant proteins.

### FIELD OF THE INVENTION

The present invention relates to a protein having proteolytic activity inducible and activable by the experimenter in the cytosol or in the secretory pathway, and uses thereof in controlling the maturation in a vital cell of a protein subject to proteolytic cleavage, and in a purification process of recombinant proteins.

### STATE OF ART

Many biological processes use protease to activate and inactivate cellular signalling cascades. In particular, viral proteases, compared to mammal proteases, are characterized by high specificity and have been widely exploited as biotechnological tools (Waugh, 2011). Thereamong, the best-known ones are 3C protease of human Rinovirus, protease of Potyvirus from Tobacco Vein Mottling Virus (TVMV) and protease of Tobacco Etch Virus (TEV) (Blommel and Fox, 2007).

The use of TEV protease in biotechnological applications has produced great interest in the last few years since its sequence specificity is much greater than that of other generally used proteases, and it further has a series of advantageous features. First of all, TEV ectopic expression in several cell lines of human origin (HeLa, HEK-293, PC12, U2OS, COS-7 and COS1) or in cultures of primary cells (neurons, astrocytes) of rodents does not induce cytotoxic effects nor causes any morphological change (Cesaratto et al., 2015; Chen et al., 2010; Wehr et al., 2006). TEV high specificity, in fact, allows it not to process any alkaline endogenous protein in the above-mentioned models, thus by making it wholly inert, very well tolerated and potentially exploitable for the development of innovative biotechnological approaches even for therapeutic purposes in mammals (Hwang et al., 2015; Jenny et al., 2003; Xiao et al., 2007). Secondly, apart from having a high proteolytic specificity, TEV is characterized by an action independent from co-factors or second messengers and by stability to physiological pH.

The scientific article with title “Directed evolution improves the catalytic efficiency of TEV protease” by Sanchez Mateo I. et al. (Nature methods, 2019) describes variants of TEV protease with replacements such as 1138T, S153N, T180A, with improved catalytic efficiency.

To date, TEV is mainly used to removed affinity “tag” from purified recombinant proteins, even if its use is always more directed to control the gene expression and processing of pro-proteins in which the cleavage sequence recognized by TEV is inserted by mutagenesis. These more recent TEV applications pave the way for the development of new therapeutic approaches based upon innovative biotechnologies, however they require a TEV whose proteolytic activity can be inducible and controllable by the experimenter.

Recently, several strategies have been developed with the purpose of controlling TEV proteolytic activity, by using: i) conditional promoters (thermal shock, galactose, light-induced promoters; Cesaratto et al. 2016); ii) activations at post-translational level (split-TEV, Dougherty et al., 1991) or iii) signal peptides to promote a localization of protease at subcellular level (Uhlmann et al., 2000; Xiao et al., 2007). In this context, it is important to underline that TEV can be used to process cytosolic as well as vesicular substrates, even if TEV expression in the endoplasmic reticulum (ER), and then in the secretory pathway, requests some specific modifications. In fact, in order to be able to address protease within the ER lumen of mammal cells it is necessary to insert a signal peptide (sec) at the N-terminal end of protein (secTEV). Moreover, in ER lumen, TEV is N-glycosylated on 4 different amino acids (N23, N52, N68 and N171) distributed in different enzyme regions. Two of them (N23 and 171) inactivate the enzyme and then are mutated to prevent N-glycosylation (N23Q, T173G); a third mutation on an exposed C (C130S) can increase TEV activity in ER lumen (secTEV-QSG) (Cesaratto et al., 2015).

Although several methods have been developed with the purpose of controlling chemically TEV proteolytic activity over time, the up-to-now used approaches have been mainly limited to the “protein complementation assay” (PCA) wherein the expression of two not functional fragments of TEV (split TEV) restore the protease enzymatic activity after association of other conjugated fusion peptides. The review article with title “Tobacco Etch Virus protease: a shortcut across biotechnologies” by Cesaratto et al. (J. Of Biotechnologym 2016) resumes the operation principles of the system based upon split TEV. The main constructs of split TEV consist of two fragments: N-TEV (1-120) and C-TEV (121-242). The reconstitution of the two fragments then is performed by means of the interaction between other fusion peptides conjugated thereto such as FKBP (FK506-binding protein) and its counterpart FRB (FKBP-rapamycin-binding). After adding rapamycin, FRB and FKBP peptides dimerize with high affinity, by inducing the re-association of the two respective TEV fragments thereto they are conjugated, thus restoring its proteolytic activity (Gray et al., 2010; Morgan et al., 2015; Williams et al., 2009). To say the truth, this approach results to be a little effective and has several limits linked to the need for co-expressing two different constructs. In fact, the variable stoichiometry in the expression of the two fragments cannot be controlled, even more in subcellular compartments such as the secretory pathway wherein many proteins are subjected to modifications which influence the function and/or localization thereof. The variable stoichiometry in the expression of the two fragments makes variable even the proteolytic response, with possible spontaneous re-assemblies of the two fragments and background activity even in absence of stimuli. In order to obviate to these limits, it was necessary to have to recourse to the use of auxiliary biotechnological strategies, based for example upon the control of split TEV accessibility to TEV recognition sequence (TEVcs), in order to limit a substrate cleavage even in absence of induction. According to a specific strategy, TEVcs cleavage sequence can be masked with Jα-helix of Light-oxygen-voltage-sensing domain 2 of Avena sativa phototropin 1 (AsLOV2) changing conformation after exposure to blue light, thus by making the substrate available only in time windows controlled by 428-474 nm-lighting (Lee et al., 2017). It follows that the system requires two stimuli to make the proteolytic cleavage happen: i) dimerization of TEV fragments and ii) exposure to blue light (Lee et al. Nat Biotechnol. 2017. doi: 10.1038/nbt.3902).

In order to try to improve the signal-background ratio, one also tried to introduce in the TEV substrates the low-affinity ENLYFQ/M sequence rather than the high affinity ENLYFQ/SX sequence. If on one side this strategy allows to mitigate the background activity, however it has the disadvantage of also reducing TEV proteolytic activity.

A reduction in the background activity of split TEV was obtained even by replacing FKBP domain in N-TEV-FKBP with a truncated version (iFKBP) which, by destabilizing more N-TEV fragment in absence of induction, reduces the possibility of spontaneous re-assembly with C-TEV-FRB (spell TEV, Dagliyan et al., 2018). The patent application WO2018/069782 A2 describes a split TEV comprising iFKBP-FRB system as dimerization domain. Although the above-mentioned approaches allow to reduce the background activity of engineered split-TEV, however the variable stoichiometry in expression of the two fragments is not controllable, even more in subcellular compartments such as the secretory pathway.

Split TEV then still nowadays has several limitations which make it not suitable for a wide in vivo use. TEV engineering based upon PCA actually has limited enormously the potential in vivo TEV applications suitable to develop new therapeutic approaches.

Other approaches for control the protease proteolytic activity are also known in literature.

The scientific article with title “Protease-based synthetic sensing and signal amplification” by Stein et al. (PNAS, 2014) and the corresponding patent application WO2014/040129 A1 describe artificially self-inhibited TVMV proteases: in particular, protease is connected to an inhibitor capable of being subjected to a conformational re-arrangement in response to the bond of a ligand molecule.

The strategy described in these documents is based upon the use of an “affinity clamp”, a two-domain artificial receptor composed of a Erbin PDZ domain connected by a serine-glycine flexible linker to a domain of engineered fibronectin of type III (FN3).

The scientific article with title “Rational design of a ligand-controlled protein conformational switch” by O. Dagliyan et al. (PNAS, 2013) further describes a “uniRapR” artificial regulatory domain, a fusion protein of iFKBP and FRB domains, wherein the bond of rapamycin to FRB determines a transition between the unfolded and folded state.

In this context, however, the need appears to be much felt for having available proteases with high specificity, the activation thereof could be induced and controlled by the experimenter in effective way, by guaranteeing a low background activity. The use of a similar engineered protease would allow to study deeply different pathophysiological processes and could pave the wave for the development of innovative therapeutic approaches.

### SUMMARY OF THE INVENTION

The object of the present invention is to provide a protease with proteolytic activity chemically inducible and controllable by the experimenter allowing to overcome the drawbacks of the systems known in the art. As previously mentioned, split TEV based upon the FRB-FKBP/rapamycin system described in the application WO2018/069782 A2, has several problems such as a heterogeneous response due to the variable stoichiometry due to the different expression of the two fragments, background activity and low activity after activation.

Differently from the split TEV constructs, the engineered protein, the invention relates to, is obtained by the insertion of an artificial regulatory domain inside the amino acid chain of protease, by making no more necessary the protein splitting into two distinct peptides (FIG. 1). This strategy allows to implement a protease characterized by chemically inducible proteolytic activity, having one single peptide chain.

Advantageously, this solution makes the protease, the invention relates to, no more subjected to a variable stoichiometry, typical of split TEV, by making the proteolytic response less heterogeneous and by reducing the background activity. In other terms, the single-chain engineered protease, the invention relates to, is not subjected to variable stoichiometry (and heterogeneous response) due to the different expression of the two fragments N-TEV-FRB or N-TEV-FKBP/C-TEV-FRB or C-TEV-FKBP. This approach is of fundamental importance for the potential applications of TEV protease in biotechnologies. The system based upon N-TEV-FRB or N-TEV-FKBP/C-TEV-FRB or C-TEV-FKBP fragments, thereto one refers in the above-mentioned documents of prior art, could not be used in subcellular compartments such as the secretory pathway indeed for the known limit of variable stoichiometry of the two fragments which would be more evident in intracellular compartments.

On the contrary, the protease the invention relates to is capable of reaching substrates localized both in the cytosol and in the vesicular compartment, result which would have been difficult to obtain by using a split-TEV-based approach. The insertion inside the protein polypeptide sequence of an artificial domain binding an activator, capable of inhibiting the proteolytic activity of said protease in the absence of said activator and of restoring the proteolytic activity thereof after addition of the activator itself, allows to control in an effective way the protease proteolytic activity.

In a preferred embodiment the invention relates to a new engineered protease of Tobacco Etch Virus (TEV), called unimolecular chemical-activatable-TEV or unica-TEV, chemically inducible and consisting of one single polypeptide chain, obtained by the insertion of one single artificial regulatory domain (uniRapR) inside the TEV amino acid chain.

The insertion of the synthetic peptide in the TEV polypeptide sequence annuls the proteolytic activity thereof, which can be restored after applying rapamycin (or non-immunosuppressive analogues) consequently by making the activity of unica-TEV controllable by the experimenter. In fact, rapamycin determines a UniRapR conformational modification capable of re-activating protease.

The possibility of using rapamycin or an inert structural analogue thereof is ideal since this molecule is capable of permeating the cellular membranes by allowing the controlled cleavage of any protein inside the cellular compartments.

It is known that UniRapR system can be used to control allosterically the kinase activity by inhibiting the ATP bond to the G-loop. In fact, the insertion of uniRapR in the catalytic site of kinases generates an instability at G-loop level in the catalytic site which makes to lose the kinase activity. However, to date, such system has not ever been used to control the activity of proteins different from kinase, nor much less to control the proteolytic activity of a protease such as TEV, in which G-loop is not present. As it results clear from the results shown in the experimental section of the present description, the authors of the present invention have demonstrated that the unica-TEV protease, apart from offering the advantage of being expressed by one single construct, shows a significant improvement in the background signal reduction (FIG. 1-3) if compared to the known split-TEV, thus proposing as valid biotechnological tool capable of overcoming the limits of the approaches known in the art. The unica-TEV, the invention relates to, keeps the advantage, shared by split TEV, of being activated by a drug, and it further has a high specificity against TEVcs cleavage sequence (FIGS. 2-3).

Thanks to these advantageous features, the protease, the invention relates to, can be used in several therapeutic applications, for example to study the molecular mechanisms at the base of maturation of proteins undergoing a proteolytic cleavage in the cytosol as well in the secretory pathway, and it can even be used in vivo in experimental models of diseases as therapeutic tool.

According to an aspect of the invention, by inserting the TEVcs recognition sequence (ENLYFQ/S) instead of the cleavage sequence recognized by endogenous protease within a protein of interest, it is possible using chemically activable unica-TEV to control at time level the maturation of such protein within the cytosol or in the secretory pathway. In particular, the new single-chain engineered TEV is capable of processing, in a way controlled by the experimenter, pro-proteins in the secretory pathway. This application can be widely used as therapeutic strategy to restore, in controlled way, the levels of mature proteins resulting to be altered in pathological contexts.

Unica-TEV then can represent a new opportunity to develop therapeutic strategies apt to contrast alteration in the levels of proteins which have to undergo maturation at neuronal level. The authors of the present invention for example have demonstrated that the unica-TEV, in the form of unica-sec-TEV, can be used effectively to control inside vital cells the maturation of proBDNF neurotrophin, having a key role in the synaptic plasticity of the neurons (see in particular the results illustrated in FIGS. 4 and 6). The insertion of TEVcs sequence in the proBDNF sequence, by replacing the site of recognition of furin/plasmin, guarantees that BNDF maturation is strictly under the experimenter's control, through the use of unica-TEV, and it is not subjected to the regulation by the endogenous proteases. The results obtained with experiments of Western blot and immunofluorescence showed that the expression levels of pro-TEVcs-BDNF are similar to those of wild-type proBDNF, suggesting that the minimum perturbation introduced in pro-TEVcs-BDNF does not influence the production or degradation thereof. Such approach allows to overcome the known limitations of the therapeutic approaches based upon the injections of BDNF as purified protein or upon the BDNF gene over-expression. Differently from other strategies, the combined use of unica-secTEV and pro-TEVcs-BDNF allow to control the maturation of these neurotrophins only in time windows and only in specific sub-populations of neurons (by using specific promoters) by avoiding adverse effects of an over-expression or an administration as purified protein. Although the BDNF cleavage sites are known, to date, the BDNF maturation control can be obtained mainly by inhibiting the endogenous protease (Furin/proprotein convertase 1/3 and tissue activator of plasminogen) by pharmacological inhibitors by obtaining a reduction in BDNF maturation instead of an increase in the mature BDNF production.

Advantageously, with the approach described in the present invention, instead, it is possible to obtain a selective activation of the system leading to the production of mature BDNF in a controlled way.

The gene codifying the unica-TEV or unica-secTEV in case can even be transferred in AAV vectors in order to develop new approaches of gene therapy to contrast even several disorders associated to alterations in the levels of produced mature pro-proteins.

In particular, in Alzheimer disease an imbalance in the processing of precursor protein of amyloid (APP) towards an amyloidogenic cleavage is noted. In neurons, the APP splitting by α-secretase ADAM10 releases the sAβPPα soluble portion and prevents the generation of senile plaques. ADAM10 is at the centre of an intense research activity since non-amyloidogenic cleavage could be of crucial importance to reduce the accumulation of Aβ oligomers which are observed in experimental models of Alzheimer's disease.

In the last years, several clinical studies which tested new treatments for Alzheimer's disease failed. The experimental approaches for Alzheimer's disease almost exclusively tried to use antibodies aiming at Aβ and tau proteins. Although these approaches failed, they were devised to cover both the familiar and sporadic forms of Alzheimer's disease. Besides, the failure in the development of new drugs effective for Alzheimer's disease is attributed, but not limited to, the highly heterogeneous nature of the disease.

Advantageously, the strategy proposed by the authors of the present invention based upon the use of the new engineered TEV, as illustrated in the experimental section (FIG. 15), could be useful for the development of new therapeutical approaches for Alzheimer's disease.

A therapeutic approach based upon the new engineered TEV is based upon the assumption that learning and memory disabilities could appear when synaptic plasticity defects occur, then by intervening on the central mechanisms of the synaptic plasticity, by promoting the production of a neurotrophin crucial for the plasticity phenomena such as BDNF. Then it is expected to safeguard some of the brain functions essential in Alzheimer's disease by overcoming the limits of the traditional therapeutic approaches. With respect to the pharmacological approaches mainly acting by inhibiting the activities of the proteins and often not sufficiently specific, the herein proposed strategy combines specificity, time control and activation rather than inhibition and, as demonstrated, it can be used on different engineered targets (by mutating the endogenous cleavage sites with the TEV cleavage sites) the expression thereof does not result to be altered in the living cells.

Then, as a whole, the present invention allows to overcome the known limits of the use of split TEV, by paving the way to a new use of protease with inducible proteolytic activity for the development of effective therapeutic approaches.

Therefore, the invention relates to:
a protein with inducible proteolytic activity comprising the polypeptide sequence of a protease and the polypeptide sequence of an activator-binding domain, wherein said domain in the absence of said activator inhibits the proteolytic activity of said protease and in the presence of said activator restores the proteolytic activity of said protease and use thereof in a treatment method;
a nucleotide sequence codifying a protein according to any one of the embodiments of the invention;
a vector for the expression of a protein with inducible proteolytic activity comprising a nucleotide sequence according to one of the embodiments of the invention;
the use of a protein with inducible proteolytic activity according to any one of the embodiments for controlling the maturation in a cell of a protein subject to proteolytic cleavage;
the use of a protein with inducible proteolytic activity according to any one of the embodiments in a purification process of recombinant proteins; and
a method for controlling the maturation in a cell of a protein subject to proteolytic cleavage comprising the following steps:
   a) co-expressing in a cell a protein with inducible proteolytic activity according to any embodiment and a protein subject to proteolytic cleavage;
   b) incubating said cell with the activators of said protein with proteolytic activity in order to induce the maturation of said protein.

Other advantages and features of the present invention will result evident from the following detailed description.

### BRIEF DESCRIPTION OF FIGURES

FIG. 1. Graphic representation of unica-TEV. TEV was split at the level of 5120-M121 in two portions, N-term (1-120) and C-term (121-236) (A). Crystallographic structure (PDB: 1LVM) of TEV, showing the UniRapR insertion site and ENLYFQ (B) substrate. Graphic representation of the 4 constructs having linkers with different length (LL: GGSGGG, ML: GGS and SL: G) (C). Then, UniRapR construct, consisting of the fusion of the sequences of the two FRB and FKBP peptide domains, was interposed, which after interaction with rapamycin changes structural conformation in order to restore the enzymatic activity of unica-TEV (D). LL—long linker, ML—medium linker, SL—small linker, NL—no linker.

FIG. 2. Split TEV has considerable levels of cleavage background in absence of activation. The strategy commonly used to control TEV enzymatic activity to date is to divide it into 2 constructs (split TEV) theoretically inactive in absence of inducer. What emerges is that after co-expression of N- and C-terminal portions respectively conjugated to FRB and FKBP domains, the protease restores the proteolytic activity even in absence of rapamycin (column 3). In particular, CFP-TEVcs-YFP cytosolic synthetic construct was used which has two fluorophores (Cerulean and Yellow Fluorescent Protein) separated by the cleavage sequence (TEVcs) recognized by TEV (ENLYFQ).

FIG. 3. Unica-TEV is capable of processing the substrate in presence of rapamycin. The use of unica-TEV allows to control the cleavage of CFP-TEVcs-YFP artificial construct in the cytosol. Western blot analyses show a complete cleavage by constitutively active TEV (column 2) and an identical cleavage profile when unica-TEV is used in presence of rapamycin (NL—no linker). The absence of cleavage background when unica-TEV is used in absence of rapamycin is important. This result highlights a great advantage in using unica-TEV with respect to the counterpart split TEV (see FIG. 2) and spell TEV (columns 3 and 4). LL—long linker, ML—medium linker, SL—small linker, NL—no linker.

FIG. 4. Control of BDNF maturation in vital cells. The use of engineered unica-secTEV allows to control the cleavage of pro-TEVcs-BDNF artificial construct in the secretory pathway (A). The analysis shows that pro-TEVcs-BDNF has an expression profile identical to proBDNF wild type (B). Moreover, analyses of Western blot (C) show the capability of unica-secTEV to make pro-TEVcs-BDNF to mature in vital cells in presence of rapamycin. The absence of cleavage background when unica-secTEV is used in absence of rapamycin is important. This result highlights a great advantage in using unica-TEV with respect to split TEV counterpart. In order to display the cleavage activity under each condition HA-pro-TEVcs-BDNF-Flag-SEP plasmid was co-expressed.

FIG. 5. Description of the strategy to purify mature BDNF from heterologous mammal cells, quantification by means of ELISA (enzyme-linked immunosorbent assay) and demonstration of a biological effect thereof.

(A) Experimental scheme: transfection of HEK293 cells with the plasmids codifying unica-secTEV and pro-TEVcs-BDNF. After 24 h, the cells were lysed to extract the proteins which were subjected to immunoprecipitation (IP) and purification by means of chromatographic columns. B) the proteins after IP were detected by means of Western blot, the production of mature BDNF around 40 kDa (BDNF+tag) in presence of rapamycin is noted. C) the immunoprecipitation with antibody binding BDNF demonstrates the capability of kit ELISA to detect the presence of proBDNF (3) and mature BDNF (4) whereas from the lysate of not transfected cells (1) or cells transfected only with unica-sec-TEV (2) neither proBDNF nor BDNF immunoprecipitates. The pro-TEVcs-BDNF is split in mature BDNF in presence of unica-sec-TEV in the transfected cells after addition of 1-4 μM rapamycin for 6 h. D) mature BNDF purified from mammal heterologous cells applied to hippocampal organotypic sections of rat induces a phosphorylation of ERK protein as shown by Western blot experiments, demonstrating that is has a biological effect.

FIG. 6. Increase in number and volume of dendritic spines after controlling BDNF maturation in vital neurons.

A) Representation of the plasmids used to transfect the brain sections containing hippocampus. B) Images of the activation effect of unica-secTEV and of the consequent BDNF maturation on the number of dendritic spines in neurons of CA1 region of organotypic sections of murine hippocampi. Graphs showing % increases in the number of dendritic spines (C) and the volume increases (D) induced by the control of BDNF maturation.

FIG. 7. Representation of the activation strategy of TMD-TEVcs-tTA system in HEK293T cells transfected with unica-TEV construct according to the present invention.

FIG. 8. The furin-plasmin splitting site in the sequence of proBDNF protein results to be highly maintained in mouse, rat and man.

FIG. 9. Comparison between the expression levels of proBDNF inserted with TEVcs (pro-TEVcs-BDNF) and expression levels of wild-type (wt) proBDNF.

FIG. 10. Determination of unica-TEV capability of splitting pro-TEVcs-BDNF in rapamycin-dependent mode.

FIG. 11. Results of in vitro protease test after immunoprecipitation of pro-TEVcs-BDNF and of unica-TEV in presence of rapamycin.

FIG. 12. Control of BDNF maturation in vital cells.

FIG. 13. The chemogenetic activation of unica-secTEV determines a clear increase in the kinase phosphorylation adjusted by the endogenous extracellular signal (ERK).

FIG. 14. Significant increase in density of dendritic spines with volume increase in the heads of dendritic spines in CA1 pyramidal hippocampus neurons together with significant changes in the morphology of dendritic spines, after activation of unica-secTEV with rapamycin for 24 h.

FIG. 15. Activation of ADAM10 disintegrin-metalloproteinase mediated by engineered TEV according to the present invention.

### DETAILED DESCRIPTION OF THE INVENTION

### Glossary

All scientific and technical terms used in the present document have the same meaning commonly meant by a person skilled in the art, except where differently indicated.

Nucleotides and amino acids are designated according to IUPAC-IUB nomenclature and/or by means of one-letter and/or three-letter code (37 C.F.R. § 1.822). The nucleotide sequences are shown only per single filament, in the direction from 5′ to 3′, from left to right.

Standard methods can be used to clone genes, to amplify and detect nucleic acids, and such techniques are known to the persons skilled in the art. See, for example, Sambrook et al., Molecular Cloning: A Laboratory Manual 2^{nd} Ed. (Cold Spring Harbor, N.Y., 1989); Ausubel et al., Current protocols in Molecular Biology (Green Publishing Associates, Inc. and John Wiley & Sons, Inc., New York), herein incorporated by reference.

Under the term “protease”, even known as “proteinase”, “peptidase” or “proteolytic enzyme”, one refers to an enzyme which is capable of catalyzing the rupture of the peptide bond between the amino group and the carboxylic group of proteins. The bond rupture mechanism provides for the use of water molecule, for this the proteases are even classified as hydrolases. The proteases can be divided based upon the structural properties of the substrate subjected to attack: the exopeptidases catalyze the removal of an amino acid on the carboxy terminal side (carboxypeptidase) or on the N-terminal end (aminopeptidase); endopeptidases catalyze the rupture of an amino acidic residue existing within the polypeptide chain and not at the ends.

Proteases can even be classified based upon the catalytic amino acidic residue used in the activation process of water molecule used to perform hydrolysis: these include serine protease, a threonine protease, cysteine protease, aspartate protease (aspartic acid), glutamate acid (glutamic acid) protease, or metalloprotease.

Proteases can be further classified based upon optimum pH for their activation: therefore, they divide into alkaline, neutral or acid proteases.

In the present description, the acronym “TEV” is used to identify, depending upon the context, “Tobacco Etch Virus” and/or “Tobacco Etch Virus nuclear-inclusion-a” endopeptidase enzyme deriving from Tobacco Etch Virus. Tobacco Etch Virus is a pathogen virus for plants belonging to the family of Potyviridae, codified by a filament of positive-polarity RNA surrounded by a capsid consisting of one single viral protein.

TEV genome is expressed entirely in one single polyprotein weighing 350 kDa, which is then cleaved by 3 endoprotease, thereamong the above-mentioned Nuclear-inclusion-A endopeptidase (NIa), better known as “TEV protease” (49 kDa). According to MEROPS classification, this protease belongs to “PA” family of C4 peptidase with structure consisting of two antiparallel β sheets (Nunn et al., 2005; Phan et al., 2002). Although homologous of serine protease, TEV protease uses a cysteine as nucleophile catalytic site.

The cleavage region recognized by TEV (TEVcs) is a specific sequence of seven amino acids: ENLYFQ-S/G, wherein proteolysis takes place between Q and S or G (Dougherty et al., 1989). TEV further includes a sequence of self-proteolysis (GHKVFM-S) in the C-terminal portion among the residues 213-219, causing a cleavage at level of M218 (Kapust et al., 2001; Parks et al., 1995). After self-proteolysis an activity loss is noted since the cleft peptide portion (219-242) inhibits the enzyme catalytic site by preventing the substrate from accessing (Nunn et al., 2005). For this reason, often a point-like mutation is introduced, truncating protease at level of V219.

The term “proteolytic activity” according to the present invention has the meaning commonly recognized in the art, that is it refers to the capability of a protein to catalyze hydrolysis of peptide bond within an amino acid sequence. Techniques aimed at assaying the proteolytic activity of a protein are known to the person skilled in the art. Such techniques include assays of enzymatic activity, for example by means of using constructs characterized by a pair of fluorophores bound therebetween by means of the specific cleavage sequence of protease under examination.

“Signal leader peptide” has the meaning commonly known in the art and it relates to short peptides (leader sequences) existing at the N-terminal end of most proteins of new synthesis which are intended towards the secretory pathway.

With the acronym “BDNF”, in the present description the brain-derived neurotrophic factor is meant, polypeptide existing at brain level in the mammals belonging to the family of neurotrophins, molecules which regulate the operation of nervous cells. The mature BDNF is active both in the central nervous system and in the peripheral system, and through TrkB receptor contributes to synaptic plasticity, to survival and to differentiation of neurons. BDNF can be found in high concentrations even in some peripheral tissues, after activation of the platelets which determines a significant increase in BDNF blood concentration. Generally, BDNF brain concentration varies depending upon the different physiological conditions (hormones, stress, food habits, physical activity, inflammatory processes), and thus it can show the presence of neurodegenerative pathologies. On the contrary, BDNF precursor, called “proBDNF”, can bind to NGF/TNFRSF16 receptor by inducing long-term synaptic depression and cell apoptosis.

As previously mentioned, the goal of the present invention is to provide an engineered protease characterized by chemically inducible proteolytic activity and controllable by the experimenter in an effective way. The strategy proposed in the present invention to provide a protease with an inducible proteolytic activity in a controlled way provides for the insertion within the polypeptide sequence of the protein of interest of an artificial domain capable of binding an activating agent. The bond with such activating agent translates into a conformational modification or change in the artificial domain which allows to restore the proteolytic activity of the protease of interest. The main advantage offered by this strategy is represented by the possibility of obtaining a protease with inducible activity characterized by one single polypeptide chain, thus overcoming the limits deriving from the use of several inactive separated constructs and/or fragments, as in case of split-TEV. Such limits include, for example, a slower catalytic activity of protease, a residual activity of background independent from induction, as well as a heterogeneous proteolytic response deriving from a variable and not controllable stoichiometry. The protein with inducible proteolytic activity according to the invention can advantageously be expressed both in the cytosol and in the secretory pathway of a cell of interest, in a way which can be controlled the experimenter, and then it can be used to control the maturation of proteins of interest which are subjected to a proteolytic cleavage within any wished cell compartment.

Then, the invention relates to a protein with inducible proteolytic activity comprising the polypeptide sequence of a protease and the polypeptide sequence of an activator-binding domain, wherein said domain in the absence of said activator inhibits the proteolytic activity of said protease and in the presence of said activator restores the proteolytic activity of said protease.

Preferably, according to an aspect of the invention, the polypeptide sequence of said activator-binding domain, could be integrated within the polypeptide sequence of the protease of interest, for example at an insertion site existing within the protease polypeptide sequence, as described hereinafter, so as to form one continuous polypeptide chain. This allows to obtain a protease with inducible proteolytic activity characterized by one single construct, or by one single polypeptide chain, by overcoming the previously mentioned limits.

According to an aspect of the invention, the integration of the peptide sequence of the activator-binding domain within the protease polypeptide sequence can take place at an internal insertion site of said protein sequence, then also determining an interruption of the protease sequence itself, provided that such insertion does not compromise to restore the protease activity after addition of an activating compound.

According to an aspect of the invention, said insertion of the polypeptide sequence del activator-binding domain can take place between a domain A and a domain B of the protease peptide sequence, wherein said domini A and B correspond to two inactive fragments of protease which can be reunited to restore the protease enzymatic activity. For example, after addition of an activating compound, the domain binding said activator be subjected to a conformational modification capable of favouring the dimerization of the two inactive domains A and B, by restoring the protease enzymatic activity.

According to an aspect of the invention, an activator-binding domain suitable to be used in the present invention then is any peptide sequence the thermal stability and/or conformation and/or distance between its N-terminal and C-terminal residues thereof depends specifically upon the bond with and/or recognition by an activating agent.

According to an aspect of the invention, said activator-binding domain is a domain constituted by the fusion of the FRB and FKBP domain. In other terms, the peptide sequence of the activator-binding domain according to the present invention can be obtained by fusing the sequences of the two FRB and FKBP peptide domains.

With the terms “FRB domain” and “FKBP domain”, the present description respectively relates to the fusion peptides known as “FKBP-rapamycin binding” and “FK506-binding protein” capable of dimerizing with high affinity after addition of rapamycin. In an embodiment of the invention, FRB and FKBP domains can be used as described in the article with title “Rational design of a ligand-controlled protein conformational switch” by O. Dagliyan et al. PNAS 2013, vol. 110, Nr. 17, herein incorporated as reference.

After interaction with the activating agent, the domain obtained by the fusion of sequences of FRB and FKBP domains is subjected to a conformational modification which allows to restore the protease enzymatic activity.

According to an embodiment, said activator-binding domain is the artificial regulating domain known with the abbreviation “uniRapR”. In a preferred embodiment of the invention, said activator-binding domain has the polypeptide sequence SEQ ID Nr. 1.

Examples of activating agents according to the present invention include compounds which are capable of recognizing and/or binding the acid sequence of said domain existing in the sequence of the protein having inducible proteolytic activity, and of causing a conformational alteration and/or modification of said domain capable of restoring the proteolytic activity of said protein. The recognition of the activating agent by the corresponding domain could take place by formation of a covalent bond or by interaction of not covalent nature, provided that it is capable of causing a moderate conformational of the peptide structure of said domain aimed at restoring the protein proteolytic activity.

According to an aspect of the invention, said activator and/or activating agent is rapamycin or an analogue thereof. Rapamycin is a macrolide antibiotic discovered as product of a bacterium (Streptomyces hygroscopicus) in a sample of ground coming from Rapa Nui. Rapamycin is also known under the name of “Sirolimus”, an immunosuppressive drug having as target in mammals a kinase threonine serine (mTOR, da mammalian Target of Rapamycin) capable of regulating growth, proliferation, motility and cell survival.

Rapamycin analogues which can be used for activating the protease, the present invention, include, for example, not immunosuppressive analogues such as for example C-16-(S)-3-methylindolerapamycin (iRap), but even DL001, AP23573, RAD001, 001-779.

According to an aspect of the present invention, the protein with inducible proteolytic activity comprises the polypeptide sequence of a protease belonging to the family of C4 peptidase. According to the MEROPS classification system of proteases, with the code “C4” one refers in particular to a family of protease with endopeptidase activity, having the cysteine as nucleophile catalytic site. From a structural point of view, the proteases belonging to the family of peptidases C4 share a central motif consisting of two antiparallel β sheets, and a histidine-aspartic acid-cysteine (His/Asp/Cys) motif, known as catalytic triad, comprised between the β sheets, wherein a histidine residue is used to activate the cysteine catalytic site by making it nucleophile.

As previously mentioned, an example of protease belonging to the family of peptidase C4 is represented by TEV protease, or Nuclear-inclusion-a endopeptidase (NIa).

In a preferred embodiment, the protein with inducible proteolytic activity according to any one of the herein described variants, comprises the polypeptide sequence of TEV protease having SEQ ID Nr. 3 or SEQ ID Nr. 4.

Depending upon if one wants to address the protease expression inside the cytosol or inside the secretory pathway, for example in the lumen of the endoplasmic reticulum (ER) of a cell, the polypeptide sequence of the protein with inducible proteolytic activity could comprise the sequence of a signal leader peptide known in the art, capable of transporting the protein attached thereto in the secretory pathway. Several signal leader sequences are known in the art and they could be selected by an expert skilled in the art depending upon protease and the target cell of interest.

In an embodiment of the invention, the protein with inducible proteolytic activity comprises the polypeptide sequence of TEV protease comprising the sequence of a signal leader peptide (sec) at the N-terminal end, having sequence MGWSLILLFLVAVATGVHS (SEQ ID Nr.: 11).

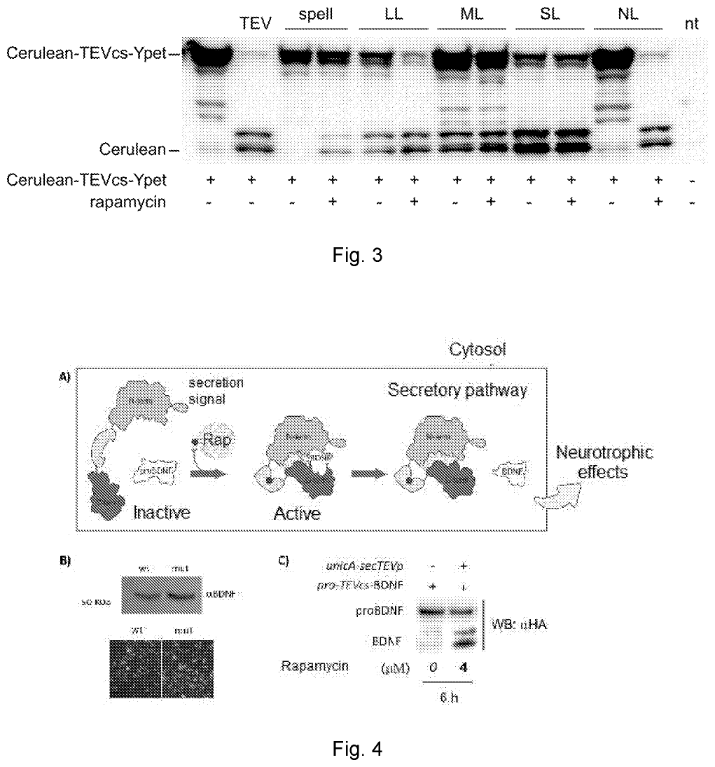

According to an aspect of the invention, the protein according to any one of the herein described embodiments can comprise one or more linker sequences for the bond between said protease and said activator-binding domain. Examples of linker sequences which can be used for the bond between the activator-binding domain and protease, include amino acid sequences having different length, for example comprising 1, 2, 3, 4, 5, or 6 amino acid residues. The most suitable linker length could be selected after experiments such as those reported in FIG. 3 of the present invention. According to an aspect of the invention said linker sequences include at least a glycine (Gly) residue. In an embodiment, said linkers have one of the following sequences: Gly-Gly-Ser-Gly-Gly-Gly (SEQ ID Nr.2), Gly-Gly-Ser and Gly.

According to an aspect of the invention, two identical linker molecules having a sequence selected among Gly-Gly-Ser-Gly-Gly-Gly (SEQ ID Nr.2), Gly-Gly-Ser or Gly can be bound at the two ends of the sequence of the activator-binding domain; the so-obtained sequence could be inserted within the peptide sequence of the protease of interest.

By means of this engineering, the protease could keep an effective and constant activity in presence of an activating agent, by offering, compared to the constitutively active counterpart, the possibility of controlling the processing of the substrate over time.

An additional aspect of the invention relates to a protein according to any one of the herein described embodiments, wherein said protease is TEV protease having SEQ ID Nr. 3 or SEQ ID Nr. 4. and wherein said domain is inserted between S120 and M121 residues with respect to SEQ ID Nr. 3 of said protease.

The protein with inducible activity according to any one of the previously described embodiments could include the polypeptide sequence of a protease characterized by the replacement and/or mutation of one or more amino acids with respect to the native acid sequence. Suitable replacements and/or mutations in the amino acid sequence of a protease are those aiming at: (1) incrementing the protease proteolytic activity, for example by speeding up the kinetics of proteolytic cleavage and/or (2) limiting the possibility of inactivating protease, for example due to N-glycosylation which typically takes place in ER lumen in mammal cells.

In a preferred embodiment, said protein having inducible proteolytic activity comprises the polypeptide sequence of a protease, wherein said protease is TEV protease having SEQ ID Nr. 3 or SEQ ID Nr. 4., and wherein in the sequence of said protease one or more of the following mutations: N23Q, T173G, C130S, 1138T, S153N and T180A with respect to the sequence of wild-type TEV protease (SEQ ID Nr. 3) are inserted. N23 and N171 glycosylation sites inactivate the enzyme, therefore N23Q and T173G mutations can be inserted at such sites to prevent N-glycosylation thereof. C130S mutation can be inserted to increase the proteolytic activity of TEV protease in ER lumen, as described in Casaratto et al. 2015 article.

The point-like mutations 1138T, S153N and T180A can be inserted in order to increase kinetics (k_{cat}/Kₘ) of engineered TEV proteolytic cleavage (unica-TEV/_{wt}TEV=2.81) (Sanchez, M. I., & Ting, A. Y., 2019). These mutations are capable of making engineered TEV protease, the invention relates to, faster than the split-TEV or TEV wild type counterpart.

In a preferred embodiment of the present invention, said protein with inducible proteolytic activity has SEQ ID Nr. 5, and it is called “unimolecular chemical-activatable TEV”, abbreviated as “unica-TEV”.

As already mentioned, in order to address TEV protease expression in the secretory pathway, or in the lumen of endoplasmic reticulum (ER) of cells of interest, it is possible to insert the sequence of a signal leader peptide (sec) at the N-terminal end of the protein, for example a signal peptide having the sequence: MGWSLILLFLVAVATGVHS (SEQ ID Nr.: 11).

The invention also relates to a protein with inducible proteolytic activity according to any one of the preceding claims, having SEQ ID Nr. 6 and called “unica-sec-TEV”.

An additional aspect of the present invention relates to a nucleotide sequence codifying a protein with inducible proteolytic activity according to any one of the previously described embodiments. According to a preferred embodiment, said nucleotide sequence codifies a protein with inducible proteolytic activity having SEQ ID Nr. 5 (unica-TEV) or SEQ ID Nr. 6 (unica-sec-TEV). In an embodiment, said nucleotide sequence has SEQ ID Nr. 9 or SEQ ID Nr. 10.

The present invention further relates to a vector for the expression of a protein with inducible proteolytic activity comprising a nucleotide sequence according to any one of the herein described embodiments. Such vector could also include a nucleotide sequence according to any one of the previously described embodiments, operatively bound to one or more regulatory sequences (for example a promoter and/or a termination sequence), allowing to control the expression, or the transcription and translation, of a protein according to any one of the embodiments of the invention in a host cell.

According to an aspect of the invention, said vector could include una nucleotide sequence according to any one of the previously described embodiments, operatively bound to (i) one or more regulatory sequences as defined above and optionally (ii) one or more nucleotide sequences and/or gene constructs such as leader sequences, selection markers, expression markers or genes and/or elements which could increase or ease the vector transformation or integration in the host cell or organism.

Examples of vectors according to the invention include DNA or RNA molecules, preferably double-stranded DNA. Vectors suitable to be used according to the present invention include vectors in a form suitable to the transformation of the host cell or organism of interest, vectors in a form suitable for the integration within the genome DNA of the host cell or the organism of interest, or still in a form suitable for the autonomous replication inside the cell or organism of interest. In particular, such vector can be a plasmid, a cosmid, YAC, a viral or transposon vector. Examples of viral vectors suitable to be used in the present description include retrovirus, adenovirus, herpes simplex, vaccine virus, and adeno-associated viruses.

A person skilled in the art, depending upon the cells and the organism of interest, will have sufficient information in the art to select the optimum vector for the wished application.

The present invention further relates to the proteins, genes and vectors described herein according to any one of the embodiments for use in a treatment method, in particular for use in a treatment method by controlling the maturation of a disease-associated protein.

The present invention further relates to the use of a protein with inducible proteolytic activity according to any one of the herein described embodiments for controlling the maturation in a cell of a protein subject to proteolytic cleavage.

According to an aspect of the invention, said protein with inducible proteolytic activity can be used for controlling the maturation of a protein subject to proteolytic cleavage in the cytosol or in other secretory pathways within a cell, for example in ER lumen of a cell.

Examples of proteins subjected to a proteolytic cleavage according to the invention, include any protein inside thereof the cleavage sequence by endogenous proteases has been changed and/or replaced by a cleavage sequence which could be recognized specifically by the protease with inducible proteolytic activity the invention relates to.

According to an embodiment, said protein subject to proteolytic cleavage is BDNF, and in particular its pro-BDNF precursor. In a preferred embodiment, said protein subject to proteolytic cleavage is pro-BDNF inside thereof the sequence of recognition by furin/serin has been replaced with the cleavage sequence of TEV protease (TEVcs, ENLYFQ, SEQ ID Nr.: 12). By way of example, said protein subject to proteolytic cleavage can be proBDNF protein codified by a mRNA having a sequence identifiable in Genback data bank by means of the following codes: M61175, M61176 or X55573, wherein the residues of recognition by furin and plasmin (MRVRRH) have been changed with TEV cleavage sequence (ENLYFQ, SEQ ID Nr.: 12). As it is clear from the results of the Western Blot and immunofluorescence experiments shown in FIG. 4, the replacement of the recognition site by furina/serin inside the sequence of pro-BDNF with the characteristic TEVcs cleavage site, allows to obtain expression levels of pro-TEVcs-BDNF protein similar to those of wild-type proBDNF, by suggesting that such modification has no impact on protein production or degradation.

Other not limiting example of proteins subjected to proteolytic cleavage include proteins which are subjected to proteolytic maturation, such as ADAM10, pro-insulin, pro-interleukin-1β, pro-orexin or many proenzymes, come for example pro-caspase, angiotensinogen, trypsinogen or plasminogen.

The invention further relates to the use of a protein with inducible proteolytic activity according to any one of the herein described embodiments in a purification process of recombinant proteins.

According to an aspect of the invention, such recombinant proteins include proteins subjected to proteolytic cleavage selected among those mentioned previously, inside thereof, for example, the cleavage sequence by endogenous proteases has been changed and/or replaced with a cleavage sequence specific for the recognition by the protease inducible the present invention relates to. The sequence of such recombinant proteins could preferably comprise a marker element, for example an affinity tag such as FLAG-tag, His-tag, strep-tag, tag-epitope or the like which allows the purification thereof by using an affinity technique. In this way, consequently to the maturation of the protein of interest thanks to the addition of an activating agent of protease with inducible proteolytic activity (for example, rapamycin), it will be possible to purify the protein of interest by affinity technique, for example by immunoprecipitation, by using a reagent capable of recognizing and/or binding said affinity tag.

An additional aspect of the invention relates to a method for controlling the maturation in a cell of a protein subject to proteolytic cleavage comprising the following steps of:
a) Co-expressing in a cell a protein with inducible proteolytic activity according to any one of the previously described embodiments and a protein subject to proteolytic cleavage;
b) Incubating said cell with the activator of said protein with proteolytic activity in order to induce the maturation of said protein.

According to an aspect of the invention, said protein subject to proteolytic cleavage is a protein inside thereof the original cleavage sequence by endogenous protease is changed and/or replaced by a specific cleavage sequence which can be recognized by the protein with inducible proteolytic activity the invention relates to.

According to a preferred embodiment, in said method, said protein subject to proteolytic cleavage is BDNF, and in particular proBDNF wherein the residues of recognition by furin and plasmin (MRVRRH) have been changed with TEV cleavage sequence (ENLYFQ, SEQ ID Nr.: 12). By way of example, in said method, said protein subject to proteolytic cleavage can be proBDNF protein codified by a mRNA having a sequence identifiable in data bank Genback by means of the following codes: M61175, M61176 o X55573, wherein the residues of recognition by furin and plasmin (MRVRRH) have been changed with TEV cleavage sequence (ENLYFQ, SEQ ID Nr.: 12).

According to an aspect of the invention, such step a) can be performed by using any technique comprised in the state of art in the field of cell biology, cell culture, gene engineering, or the like, as well as by using any of the previously described nucleotide sequences or vectors. Preferably, according to an aspect of the invention, in said method, said cell is a mammal cell.

As the features of the protein with used inducible proteolytic activity are known, the person skilled in the art could use in step b) of the herein described method an adequate activating compound selected among the previously described compounds.

According to an additional aspect of the invention, said method comprises an additional step c) of detecting the mature shape of said protein obtainable with step b). Such detection can be performed by using any technique known in the art, for example by means of electrophoretic assay, immuno-enzymatic assay, colorimetric assay.

According to a preferred embodiment, such step c) of detecting the mature protein can be performed by means of electrophoretic analysis on gel of SDS-polyacrylamide.

The invention also relates to a protein o nucleotide sequence or vector according to any one of the herein described embodiments for use in a treatment method, in particular for use in a treatment method for controlling the maturation of a disease-associated protein.

### EXAMPLES

Materials and Methods

The methods for obtaining engineered TEV as well as its functional validation comprise:
Use of specific sets of primers described in the section “List of sequences”;
Cloning plasmids and mutagenesis by “QuikChange site-directed mutagenesis kit” of Agilent Technologies, Inc;
Polymerase chain reaction (PCR);
Transformation of competent cells;
DNA sequencing;
Transfection of heterologous cells;
Enzymatic reaction;
Western blot.

In the specific case, for each mutagenesis the following protocol was used:
1) Add 35.5 μl of bi-distilled H₂O 5 μl of 10× reaction buffer (Agilent Technologies); 5 μl dNTP mix (stock 10 mM); 1.25 μl primer forward (stock 20 μM); 1.25 μl primer reverse (stock 20 μM); 1 μl of plasmid 0.1 μg/μl; 1 μl of PfuTurbo DNA polymerase (Agilent Technologies);
2) PCR:
   a) 95° C. for 30 s;
   b) 95° C. for 30 s;
   c) 55° C. for 60 s;
   d) 72° C. for 1 min/kb of plasmid;
Repeat b, c, d for 16 cycles;
3) Add 1 μl of Dpnl restriction endonuclease (Agilent Technologies) and incubate at 37° C. for 1 h;
4) Use 2 μl of final solution to transform the competent cells;
5) Select some colonies, extract the plasmid and sequencing.

In order to engineer each one of the two TEVs and insert “UniRapR” construct, Gibson Assembly was performed. At first 2 distinct PCRs were performed:
6) In the first PCR the solution 1) was used, with specific primers, with TEV template DNA and following reaction conditions:
   a) 95° C. for 30 s;
   b) 95° C. for 30 s;
   c) 66° C. for 60 s;
   d) 72° C. for 1 min/kb of plasmid;
      Repeat b, c, d for 25 cycles;
7) In the second PCR the solution 1) was used, with specific primers, with UniRapR template DNA with the following reaction conditions:
   e) 95° C. for 30 s;
   f) 95° C. for 30 s;
   g) 72° C. for 60 s;
   h) 72° C. for 1 min/kb of plasmid;
      Repeat b, c, d for 25 cycles;
8) Load part of PCR product with 0.7% agarose gel and separate DNA by electrophoresis with the purpose of evaluating the amplification quality;
   PCR products are 6783 bp for open TEV plasmid, 6213 bp for open sec-TEV plasmid and ˜600 for UniRapR fragment to be inserted.
9) Add 1 μl of Dpnl restriction endonuclease (Agilent Technologies) and incubate at 37° C. for 1 h;
10) Prepare Gibson Assembly reaction; add 6 μl of bi-distilled H₂O 2 μl of PCR amplification coming from TEV plasmid and 2 μl of PCR amplification coming from the amplified UniRap fragment. Add the so-obtained 10 μl to Master Mix Gibson Assembly (Gibson Assembly® Cloning Kit, NEB). Incubate 1 h at 50° C.
11) Use 2 μl of the final solution to transform the competent cells;
12) Select some colonies, extract the plasmid and sequencing;

Enzymatic Activity of Unica-TEV in Vital Cells
13) Use CFP-TEVcs-YFP synthetic recombinant protein as substrate;

This artificial construct has two fluorophores conjugated by the cleavage sequence recognized by TEV (TEVcs). AntiGFP (Biolegend) antibody was used to verify cleavage of CFP-TEVcs-YFP by unica-TEV.

In order to do it, HEK293T cells were transfected (PEI MAX—Polysciences, Inc.) with the plasmids codifying constitutively active wild-type TEV (pcDNA-TEV), and engineered TEV with UniRapR (pcDNA-unica-TEV) and left to grow for 24-48 h at 37° C., 5% CO₂.

For both of them, CFP-TEVcs-YFP was co-transfected.

After 24h the cells were treated for 1-3 h with rapamycin (1 μM) or DMSO as control. The cells were washed with cold PBS and lysed with lysis buffer containing rapamycin (1 μM) or DMSO. 10% of the volume of samples was prepared (addition of 2× Laemmli protein sample buffer+boiling for 5 min) for the electrophoretic analysis on gel of SDS-polyacrylamide.

Enzymatic activity of unica-secTEV in vital cells Use pro-TEVcs-BDNF human recombinant as substrate;

ProBDNF was mutagenized with the purpose of having, instead of canon cleavage sequence (MRVRRH) the cleavage sequence (TEVcs) recognized by TEV (ENLYFQ). AntiHA (Biolegend) antibody was used to verify pro-TEVcs-BDNF cleavage in mature BDNF by unica-TEV.

In order to do it, HEK293T cells were transfected (PEI MAX—Polysciences, Inc.) with the plasmids which codify for constitutively active wild type TEV (pcDNA-secTEV-SV5), and engineered unica-TEV (pcDNA-unica-secTEV-SV5) and left to grow for 24-48 h at 37° C., 5% CO₂.

For both of them pro-TEVcs-BDNF (HA-pro-TEVcs-BDNF-Flag-SEP) was co-transfected.

After 24h the cells were then treated for 1-3 h with rapamycin (1 μM) or DMSO as control. The cells were washed with cold PBS and lysed with lysis buffer containing rapamycin (1 μM) or DMSO. 10% of volume of samples was prepared (addition of 2× Laemmli protein sample buffer+boiling for 5 min) for the electrophoretic analysis on gel of SDS-polyacrylamide.

Production of Purified BDNF

HEK293T cells were transfected (PEI MAX—Polysciences, Inc.) with the plasmids codifying for unica-secTEV (pcDNA-unica-secTEV-SV5) and pro-TEVcs-BDNF (HA-pro-TEVcs-BDNF-Flag-SEP) and left for 24 h at 37° C., 5% CO₂.

After 24 h the cells were treated for 1-3 h with rapamycin (1 μM) or DMSO as control. The cells were washed with cold PBS and lysed with lysis buffer containing rapamycin (1 μM) or DMSO. The lysate was immunoprecipitated with FLAG-M2-beads for 1 h and subsequently FLAG peptide (sigma F3290-4 mg) was added. After 1 h the beads were removed by Micro bio-spin Colums®—Bio-Rad. The eluted protein was kept at −20° C. Once quantified by commercially available ELISA kits its biological effect was tested as described in FIG. 6.

### Example 1—Determination of Activity of Unica-TEV in HEK293T Cells

A system was used based upon the expression of a fluorophore after nuclear translocation of a transactivator sequestered on the plasmatic membrane by a TEV recognition site (TEVcs).

In particular, the inventors made to express in HEK293T cells a “tetracycline operator EGFP conjugated” (tetO-EGFP) and a synthetic protein consisting of a transactivator controlled by tetracycline (tTA), bound to a transmembrane domain (TMD) through a TEV recognition site (TEVcs) (TMD-TEVcs-tTA) (FIG. 7).

The activation of uniRapR (also called: unica-TEV) mediated by rapamycin split tTA which translocated into the nucleus and started the EGFP expression. Twenty-four hours later a robust increase in the EGFP expression was noted in HEK293T cells treated with rapamycin and transfected with all unica-TEV constructs (FIG. 7). However, in the control cells exposed to the vehicle, EGFP signal was substantially lower in the cells transfected with unica-TEV without linker.

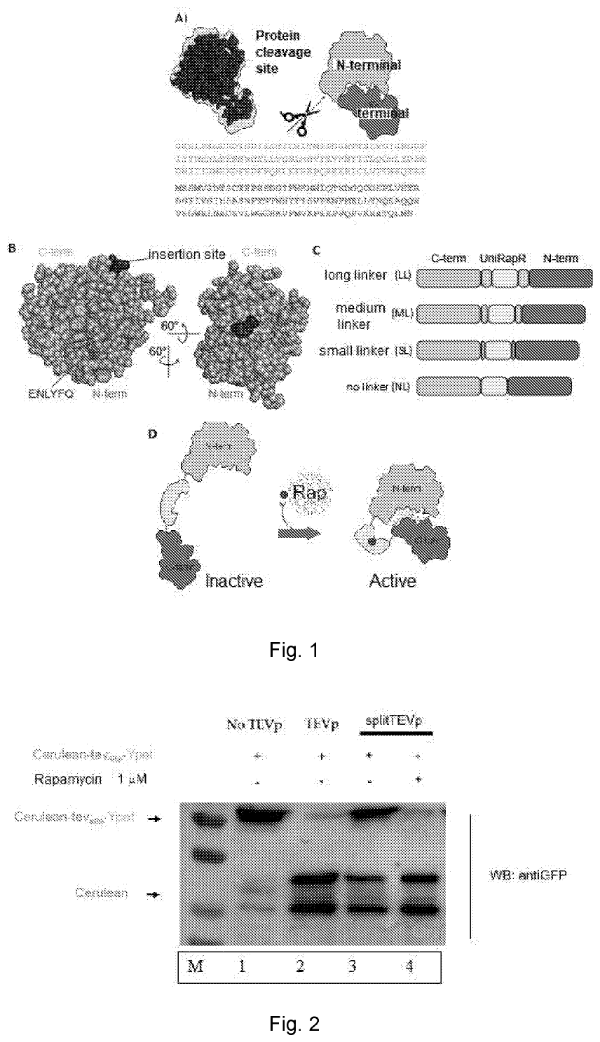

As already mentioned, the background activity of the system based upon split TEV is a known problem in literature, so much so that in order to mitigate the background activity of fragmented TEV it was necessary, for example, to mask TEV recognition sequence (TEVcs) with AsLOV2 (Jα-helix of Avena sativa phototropin 1 light-oxygen-voltage 2 domains) which is released only after exposition to blue light. Lee and colleagues evaluated the operation of the system based upon C-TEV/N-TEV/AsLov2 on EGFP gene expression (FIG. 1 a, d).

In case of the present invention, the only application of permeable molecules (rapamycin) allows to obtain the transactivator cleavage, whereas in case of NL construct (no linker), in absence of activator, EGFP expression is almost wholly absent by confirming that the new single-peptide chain engineered TEV exceeds the limits of background activity of the system based upon C-TEV/N-TEV.

### Example 2—Application of Engineered TEV System for Inducible BDNF Maturation

The secreted proteolytic splitting products, as pre-proproteins, are synthetized on the rough endoplasmic reticulum (ER). The pre-sequence peptide directs the synthesis of pro-proteins towards ER wherein the peptide pre- is split immediately. Then the pro-proteins translocate from Golgi apparatus to trans-Golgi network (TGN) wherein the pro-domain is separated to provide the mature products. From TGN, the mature products can be released continuously in absence of any triggering stimulus or released in response to extra-cell triggering events raising the Ca2+ intracellular concentration. For example, BDNF is released continuously by TGN with small vesicle granules in Ca2+-independent mode, but it can even be released by bigger vesicles on inflow of Ca2+ induced by neuronal depolarization. The mature BDNF is produced by proBDNF proteolytic splitting, catalyzed along the secretory route by protease Furin/proprotein convertasi 1/3 (PC1/3), and at extracellular level through the tissue activator of plasminogen (tPA)/cascade of plasminogen. By activating the tropomyosin kinase-B (TrkB) receptors, BDNF plays a key role in the formation and maturation of synapses, in synaptic plasticity, in survival and differentiation of neural staminal cells. In order to make BDNF maturation inducible, TEVcs was inserted in proBDNF sequence by replacing Furin/Plasmin splitting site (FIGS. 8 and 9). This site was selected since it is highly preserved in mouse, rat and man.

The expression levels of this proBDNF inserted with TEVcs (pro-TEVcs-BDNF) were similar to those of wild-type (wt) proBDNF, by suggesting that this replacement of small sequences does not influence on the protein production and degradation (FIG. 9).

Then, unica-TEV capability of splitting pro-TEVcs-BDNF in rapamycin-dependent mode was evaluated. As provided, nor unica-TEV in presence of rapamycin, nor an active mutating form of TEV, split pro-TEVcs-BDNF in the living cells (that is HEK293T) due to the cytosolic localization of TEV protease (FIG. 10).

However, by performing an in vitro protease test after immunoprecipitation of pro-TEVcs-BDNF and of unica-TEV in presence of rapamycin, SDS-PAGE migration of pro-peptide was noted (FIG. 11).

These data showed unica-TEV capability of cleaving pro-TEVcs-BDNF, by confirming that the splitting in the living cells depends upon the different localization of pro-TEVcs-BDNF and upon the engineered protease.

In order to make unica-TEV functionally active along the secretion route of the mammal cells (unica-secTEV), the secretion signal was added to the N-terminal and three mutations were introduced as described in the preceding experimental sections. When HEK293T cells expressing unica-secTEV and pro-TEVcs-BDNF were treated with rapamycin, a 41-kDa band was noted designating BDNF maturation in the living cells (FIG. 12). This result demonstrates that the unica-secTEV variant can be activated in robust way by rapamycin inside the secretory pathway.

In order to evaluate if mature BDNF obtained after activation of unica-secTEV was able to bind and activate its TrkB endogenous target receptor, pro-TEVcs-BDNF was expressed, unica-secTEV with TrkB-mGFP. The chemogenetic activation of unica-secTEV led to a clear increase in the kinase phosphorylation regulated by the endogenous extracellular signal (ERK) (FIG. 13).

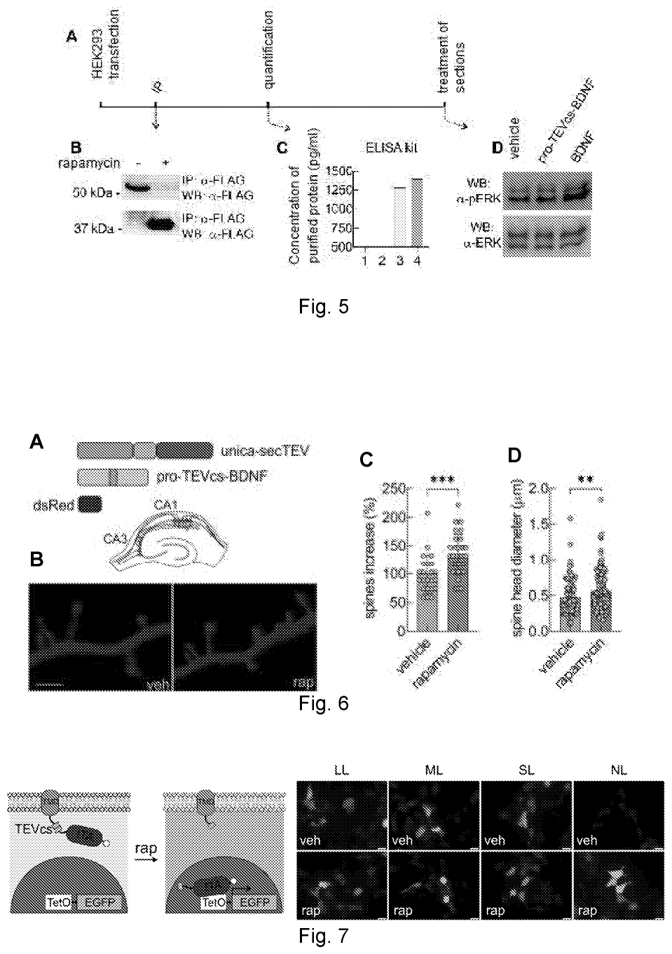

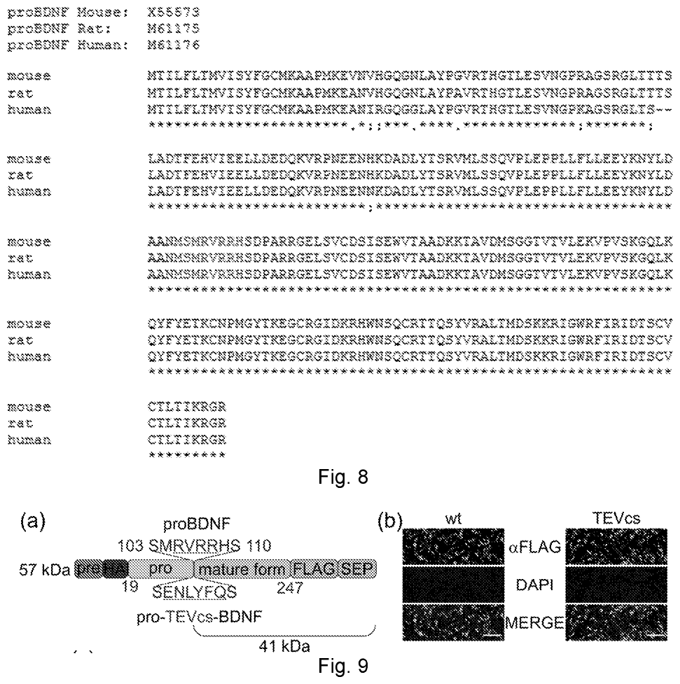

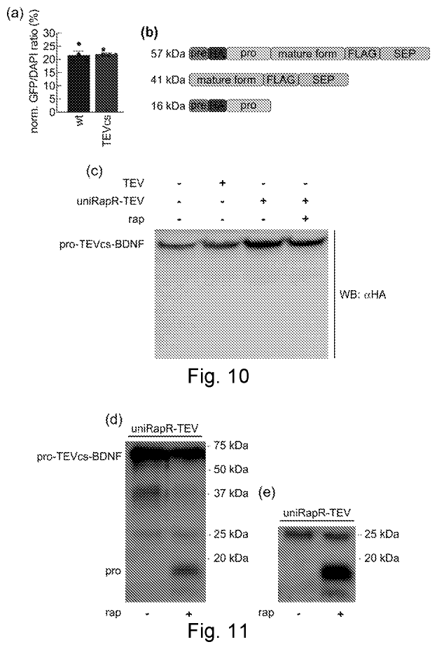

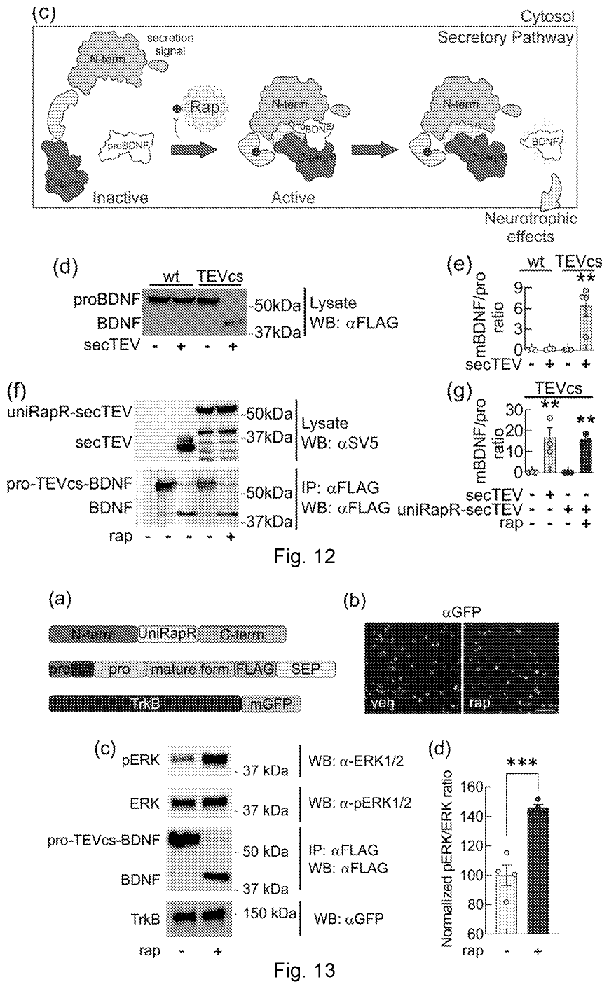

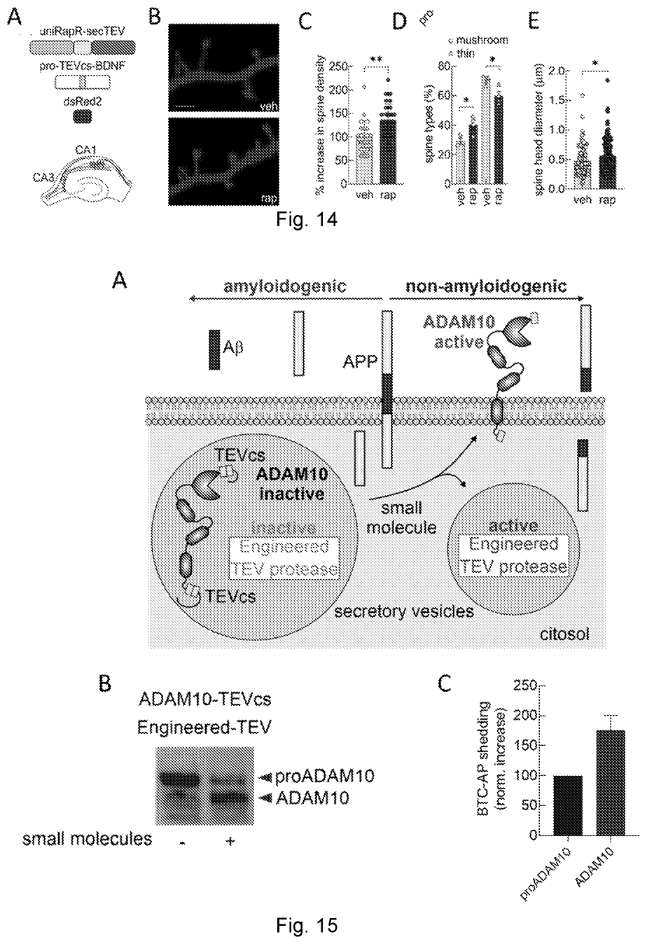

In order to test the biological activity of mature BDNF obtained with the chemogenetic strategy in neurons, BDNF was immunoprecipitated and purified by HEK293T cells co-transfected with unica-secTEV and treated with rapamycin. The hippocampus organo-typical sections treated for 30 minutes with this purified mature BDNF showed a significant increase in ERK phosphorylation signals (FIG. 5). These data showed that the minimum perturbation introduced in BDNF did not influence the biological activity of mature BDNF. Although it was demonstrated that BDNF supports a variety of functions of dendritic spines, including their maturation and plasticity, the exact contribution of mature BDNF secreted by the pre-counter postsynaptic sites is still under discussion. BDNF presynaptic release contributes to its paracrine actions, whereas it was suggested that its postsynaptic secretion is responsible for the autocrine effects. In fact, BDNF biochemical features, including the positive charges on its surface, prevent the spreading thereof and keep its action locally at level of synapses. This feature obstructs even the effectiveness of injections of purified BDNF as therapeutic strategy. Since the extracellular application could involve both sides, even the exogenous application of purified BDNF is not useful in sectioning the roles of pre- and postsynaptic release. However, BDNF gene deletion, in particular in CA3 or CA1 neurons, revealed that the paracrine release affects the force of synaptic plasticity, whereas BDNF autocrine signalling contributes to maintain the synaptic enhancement. These gene approaches involve the deletion both of proBDNF and BDNF, the contribution of each form to the observed effect cannot be distinguished. The chemogenetic strategy developed by the inventors to control the protein cleavage is useful even to evaluate the specific autocrine action of BDNF inducible intracellular maturation in CA1 pyramidal neurons. In order to monitor BDNF specific effects in the postsynaptic neurons, organo-typical sections containing the hippocampus of rats with unica-secTEV, pro-TEVcs-BDNF and dsRed2 were transfected ballistically to display in a fluorescent way CA1 pyramidal neurons. After 48 h, unica-secTEV with rapamycin was activated for 24 h. A significant increase in the density of dendritic spines was noted with an increase in the volume of heads of dendritic spines in CA1 pyramidal hippocampus neurons together with significant changes in the morphology of dendritic spines (FIG. 14).

These data showed that BDNF spontaneous release induced an autocrine trophic effect in CA1 pyramidal neurons independently from the induction of synaptic plasticity. A new strategy was then generated allowing a complete splitting of the proteins of interest in the subcellular compartments, which allowed us to create and test in an inducible way proteolytical splitting products.

### Example 3—Application of Engineered TEV System for ADAM10 Activation

The previously illustrated strategy was applied by the authors of the present invention even to other target proteins. Thereamong, ADAM10 disintegrin-metalloproteinase which stands out as particularly critical neuronal protein since it catalyses the non-amyloidogenic splitting of the amyloid precursor protein (APP) by α-secretase critically involved in the pathogenesis of Alzheimer's disease. In line with the previously illustrated strategy, the inventors devised ADAM10 by replacing its endogenous protease sequence with TEV recognition site (TEVcs). This change led to ADAM10 activation mediated by engineered TEV of the invention in the living cells without influencing ADAM10 expression. The capability of the so-obtained mutating ADAM 10 to be controlled space-temporally by using betacellulin (ADAM10 substrate) bound to an alkaline phosphatase (AP) was demonstrated. The activation of single-chain engineered TEV protease according to the present invention, in the secretory pathway, induced the selective cleavage of betacellulin and AP release from HEK293T cells which reflects the capability of the herein proposed strategy of controlling ADAM10 activity (FIG. 15). To the knowledge of the inventors, this is the only strategy allowing a selective activation of ADAM10 in vital cells.

### LIST OF SEQUENCES IN THE DESCRIPTION

SEQ ID Nr.: 1 Peptide sequence of activator-binding domain
TCVVHYTGMLEDGKKFDSSRDRNKPFKFMLGKQEVIRGWEEGVAQMSVGQRAKLTISPDYAYGA
TGHGSGSGSGVKDLLQAWDLYYHVFRRISGPPGPGSGLWHEMWHEGLEEASRLYFGERNVKG
MFEVLEPLHAMMERGPQTLKETSFNQAYGRDLMEAQEWCRKYMKSGSSGGSGSGIIPPHATLVF
DVELLKLE
SEQ ID Nr.: 2 Peptide sequence of a linker molecule
GGSGGG
SEQ ID Nr.: 3 Peptide sequence of wild-type TEV protease
GESLFKGPRDYNPISSTICHLTNESDGHTTSLYGIGFGPFIITNKHLFRRNNGTLLVQSLHGVFKVK
NTTTLQQHLIDGRDMIIIRMPKDFPPFPQKLKFREPQREERICLVTTNFQTKSMSSMVSDTSCTFPS
SDGIFWKHWIQTKDGQCGSPLVSTRDGFIVGIHSASNFTNTNNYFTSVPKNFMELLTNQEAQQWW
SGWRLNADSVLWGGHKVFMVKPEEPFQPVKEATQLMN
SEQ ID Nr.: 4 Peptide sequence of sec-TEV protease
MGWSLILLFLVAVATGVHSQGESLFKGPRDYNPISSTICHLTQESDGHTTSLYGIGFGPFIITNKHLF
RRNNGTLLVQSLHGVFKVKNTTTLQQHLIDGRDMIIIRMPKDFPPFPQKLKFREPQREERICLVTTN
FQTKSMSSMVSDTSSTFPSSDGIFWKHWIQTKDGQCGSPLVSTRDGFIVGIHSASNFGNTNNYFT
SVPKNFMELLTNQEAQQWSGWRLNADSVLWGGHKVFMSKPEEPFQPVKEATQLMNEGGLE
SEQ ID Nr.: 5 Peptide sequence of unica-TEV engineered protease
GESLFKGPRDYNPISSTICHLTNESDGHTTSLYGIGFGPFIITNKHLFRRNNGTLLVQSLHGVFKVK
NTTTLQQHLIDGRDMIIIRMPKDFPPFPQKLKFREPQREERICLVTTNFQTKSGGSTCVVHYTGML
EDGKKFDSSRDRNKPFKFMLGKQEVIRGWEEGVAQMSVGQRAKLTISPDYAYGATGHGSGSGS
GVKDLLQAWDLYYHVFRRISGPPGPGSGLWHEMWHEGLEEASRLYFGERNVKGMFEVLEPLHA
MMERGPQTLKETSFNQAYGRDLMEAQEWCRKYMKSGSSGGSGSGIIPPHATLVFDVELLKLEGG
SMSSMVSDTSCTFPSSDGTFWKHWIQTKDGQCGNPLVSTRDGFIVGIHSASNFTNTNNYFASVP
KNFMELLTNQEAQQWWSGWRLNADSVLWGGHKVFMVKPEEPFQPVKEATQLMN
SEQ ID Nr.: 6 Peptide sequence of unica-sec-TEV engineered protease
MGWSLILLFLVAVATGVHSQGAQGESLFKGPRDYNPISSTICHLTQESDGHTTSLYGIGFGPFIITN
KHLFRRNNGTLLVQSLHGVFKVKNTTTLQQHLIDGRDMIIIRMPKDFPPFPQKLKFREPQREERICL
VTTNFQTKSGGSTCVVHYTGMLEDGKKFDSSRDRNKPFKFMLGKQEVIRGWEEGVAQMSVGQR
AKLTISPDYAYGATGHGSGSGSGVKDLLQAWDLYYHVFRRISGPPGPGSGLWHEMWHEGLEEA
SRLYFGERNVKGMFEVLEPLHAMMERGPQTLKETSFNQAYGRDLMEAQEWCRKYMKSGSSGG
SGSGIIPPHATLVFDVELLKLEGGSMSSMVSDTSSTFPSSDGTFWKHWIQTKDGQCGNPLVSTRD
GFIVGIHSASNFGNTNNYFASVPKNFMELLTNQEAQQWWSGWRLNADSVLWGGHKVFMSKPEE
PFQPVKEATQLMNEGGLE
SEQ ID Nr.: 7 Nucleotide sequence of wild-type TEV protease
ATGGGAGAAAGCTTGTTTAAGGGGCCGCGTGATTACAACCCGATATCGAGCACCATTTGTCAT
TTGACGAATGAATCTGATGGGCACACAACATCGTTGTATGGTATTGGATTTGGTCCCTTCATC
ATTACAAACAAGCACTTGTTTAGAAGAAATAATGGAACACTGTTGGTCCAATCACTACATGGTG
TATTCAAGGTCAAGAACACCACGACTTTGCAACAACACCTCATTGATGGGAGGGACATGATAA
TTATTCGCATGCCTAAGGATTTCCCACCATTTCCTCAAAAGCTGAAATTTAGAGAGCCACAAAG
GGAAGAGCGCATATGTCTTGTGACAACCAACTTCCAAACTAAGAGCATGTCTAGCATGGTGTC
AGACACTAGTTGCACATTCCCTTCATCTGATGGTATATTCTGGAAGCATTGGATTCAAACCAAG
GATGGGCAGTGTGGCAGTCCATTAGTATCAACTAGAGATGGGTTCATTGTTGGTATACACTCA
GCATCGAATTTCACCAACACAAACAATTATTTCACAAGCGTGCCGAAAAACTTCATGGAATTGT
TGACAAATCAGGAGGCGCAGCAGTGGGTTAGTGGTTGGCGATTAAACGCTGACTCAGTATTG
TGGGGGGGCCATAAAGTTTTCATGGTGAAACCTGAAGAACCTTTTCAGCCAGTTAAGGAAGC
GACTCAACTCATGAAT
SEQ ID Nr.: 8 Nucleotide sequence of sec-TEV protease
ATGGGCTGGAGCCTGATCCTCCTGTTCCTCGTCGCTGTGGCTACAGGTGTGCACTCTCAGatg
GGGGAAAGCCTGTTCAAGGGACCAAGGGACTACAATCCAATCTCCTCAACTATCTGCCACCT
GACTcAgGAAAGCGACGGACATACCACATCTCTGTACGGAATTGGCTTCGGGCCCTTCATCAT
TACTAACAAGCACCTGTTTCGGAGAAACAATGGCACCCTGCTGGTGCAGAGTCTGCACGGGG
TGTTCAAGGTCAAAAATACTACCACACTGCAGCAGCATCTGATTGACGGACGAGATATGATCA
TTATCCGGATGCCAAAGGACTTCCCCCCTTTTCCCCAGAAGCTGAAGTTCCGGGAGCCCCAG
AGGGAGGAACGCATCTGCCTGGTGACTACCAACTTCCAGACCAAATCCATGAGCTCCATGGT
CTCCGACACCTCTTcTACATTCCCTTCTAGTGATGGCATCTTCTGGAAGCACTGGATCCAGAC
AAAAGACGGACAGTGCGGCAGTCCACTGGTGTCAACCAGAGATGGGTTTATTGTCGGAATCC
ATTCAGCCAGCAACTTCggAAATACTAACAATTACTTCACCTCTGTGCCCAAAAACTTCATGGA
GCTGCTGACTAATCAGGAAGCACAGCAGTGGGTGAGCGGATGGCGCCTGAATGCTGATTCC
GTGCTGTGGGGCGGGCATAAGGTCTTCATGAGCAAACCTGAAGAGCCATTTCAGCCCGTCAA
GGAAGCCACCCAGCTGATGAaCGAAggGGgCctggaA
SEQ ID Nr.: 9 Nucleotide sequence of unica-TEV engineered protease
ATGGGAGAAAGCTTGTTTAAGGGGCCGCGTGATTACAACCCGATATCGAGCACCATTTGTCAT
TTGACGAATGAATCTGATGGGCACACAACATCGTTGTATGGTATTGGATTTGGTCCCTTCATC
ATTACAAACAAGCACTTGTTTAGAAGAAATAATGGAACACTGTTGGTCCAATCACTACATGGTG
TATTCAAGGTCAAGAACACCACGACTTTGCAACAACACCTCATTGATGGGAGGGACATGATAA
TTATTCGCATGCCTAAGGATTTCCCACCATTTCCTCAAAAGCTGAAATTTAGAGAGCCACAAAG
GGAAGAGCGCATATGTCTTGTGACAACCAACTTCCAAACTAAGAGCggtggatcaacctgcgtggtgcac
tacaccgggatgcttgaagatggaaagaaatttgattcctcccgggacagaaacaagccctttaagtttatgctaggcaagcaggaggtg
atccgaggctgggaagaaggggttgcccagatgagtgtgggtcagagagccaaactgactatatctccagattatgcctatggtgccact
gggcacggttcgggctccggatcaggcgtcaaggacctcctccaagcctgggacctctattatcatgtgttccgacgaatctcaggtcctcc
aggacctggatcaggtctctggcatgagatgtggcatgaaggcctggaagaggcatctcgtttgtactttggggaaaggaacgtgaaagg
catgtttgaggtgctggagcccttgcatgctatgatggaacggggcccccagactctgaaggaaacatcctttaatcaggcctatggtcga
gatttaatggaggcccaagagtggtgcaggaagtacatgaaatcagggtcatcagggggctccggatcaggcatcatcccaccacatg
ccactctcgtcttcgatgtggagcttctaaaactggaaggtggatcaATGTCTAGCATGGTGTCAGACACTAGTTGCA
CATTCCCTTCATCTGATGGTACGTTCTGGAAGCATTGGATTCAAACCAAGGATGGGCAGTGTG
GCAATCCATTAGTATCAACTAGAGATGGGTTCATTGTTGGTATACACTCAGCATCGAATTTCAC
CAACACAAACAATTATTTCGCAAGCGTGCCGAAAAACTTCATGGAATTGTTGACAAATCAGGA
GGCGCAGCAGTGGGTTAGTGGTTGGCGATTAAACGCTGACTCAGTATTGTGGGGGGGCCAT
AAAGTTTTCATGGTGAAACCTGAAGAACCTTTTCAGCCAGTTAAGGAAGCGACTCAACTCATG
AAT
SEQ ID Nr.: 10 Nucleotide sequence of unica-sec-TEV engineered protease
ATGGGCTGGAGCCTGATCCTCCTGTTCCTCGTCGCTGTGGCTACAGGTGTGCACTCTCAGGG
CGCGCAAGGGGAAAGCCTGTTCAAGGGACCAAGGGACTACAATCCAATCTCCTCAACTATCT
GCCACCTGACTcAgGAAAGCGACGGACATACCACATCTCTGTACGGAATTGGCTTCGGGCCCT
TCATCATTACTAACAAGCACCTGTTTCGGAGAAACAATGGCACCCTGCTGGTGCAGAGTCTGC
ACGGGGTGTTCAAGGTCAAAAATACTACCACACTGCAGCAGCATCTGATTGACGGACGAGAT
ATGATCATTATCCGGATGCCAAAGGACTTCCCCCCTTTTCCCCAGAAGCTGAAGTTCCGGGAG
CCCCAGAGGGAGGAACGCATCTGCCTGGTGACTACCAACTTCCAGACCAAATCCggtggatcaac
ctgcgtggtgcactacaccgggatgcttgaagatggaaagaaatttgattcctcccgggacagaaacaagccctttaagtttatgctaggc
aagcaggaggtgatccgaggctgggaagaaggggttgcccagatgagtgtgggtcagagagccaaactgactatatctccagattatg
cctatggtgccactgggcacggttcgggctccggatcaggcgtcaaggacctcctccaagcctgggacctctattatcatgtgttccgacga
atctcaggtcctccaggacctggatcaggtctctggcatgagatgtggcatgaaggcctggaagaggcatctcgtttgtactttggggaaag
gaacgtgaaaggcatgtttgaggtgctggagcccttgcatgctatgatggaacggggcccccagactctgaaggaaacatcctttaatca
ggcctatggtcgagatttaatggaggcccaagagtggtgcaggaagtacatgaaatcagggtcatcagggggctccggatcaggcatc
atcccaccacatgccactctcgtcttcgatgtggagcttctaaaactggaaggtggatcaATGAGCTCCATGGTCTCCGACA
CCTCTTcTACATTCCCTTCTAGTGATGGCAcCTTCTGGAAGCACTGGATCCAGACAAAAGACG
GACAGTGCGGCAaTCCACTGGTGTCAACCAGAGATGGGTTTATTGTCGGAATCCATTCAGCCA
GCAACTTCggAAATACTAACAATTACTTCgCCTCTGTGCCCAAAAACTTCATGGAGCTGCTGAC
TAATCAGGAAGCACAGCAGTGGGTGAGCGGATGGCGCCTGAATGCTGATTCCGTGCTGTGG
GGCGGGCATAAGGTCTTCATGAGCAAACCTGAAGAGCCATTTCAGCCCGTCAAGGAAGCCAC
CCAGCTGATGAaCGAAggGGgCctggaA
SEQ ID Nr.: 11 Peptide sequence of the signal leader peptide
MGWSLILLFLVAVATGVHS
SEQ ID Nr.: 12 Cleavage peptide sequence of TEV protease
ENLYFQ

List of Primers Used to Implement Mutageneses

SEQ ID Nr.: 13 Nucleotide sequence of primer RV TEV_I138T
CCAATGCTTCCAGAACGTACCATCAGATGAAGGGAATGTGCAA
SEQ ID Nr.: 14 Nucleotide sequence of primer FW TEV_I138T
TTGCACATTCCCTTCATCTGATGGTACGTTCTGGAAGCATTGG
SEQ ID Nr.: 15 Nucleotide sequence of primer RV TEV_S153N
TTGATACTAATGGATTGCCACACTGCCCATCCTTG
SEQ ID Nr.: 16 Nucleotide sequence of primer FW TEV_S153N
CAAGGATGGGCAGTGTGGCAATCCATTAGTATCAA
SEQ ID Nr.: 17 Nucleotide sequence of primer RV TEV_T180A
GTTTTTCGGCACGCTTGCGAAATAATTGTTTGTGTTGGTGAA
SEQ ID Nr.: 18 Nucleotide sequence of primer FW TEV_T180A
TTCACCAACACAAACAATTATTTCGCAAGCGTGCCGAAAAAC
SEQ ID Nr.: 19 Nucleotide sequence of primer RV secTEV_I138T
CCAGTGCTTCCAGAAGGTGCCATCACTAGAAGG
SEQ ID Nr.: 20 Nucleotide sequence of primer FW secTEV_I138T
CCTTCTAGTGATGGCACCTTCTGGAAGCACTGG
SEQ ID Nr.: 21 Nucleotide sequence of primer RV secTEV_S153N
GACACCAGTGGATTGCCGCACTGTCCGTC
SEQ ID Nr.: 22 Nucleotide sequence of primer FW secTEV_S153N
GACGGACAGTGCGGCAATCCACTGGTGTC
SEQ ID Nr.: 23 Nucleotide sequence of primer RV secTEV_T180A
GTTTTTGGGCACAGAGGCGAAGTAATTGTTAGTATTTCCGA
SEQ ID Nr.: 24 Nucleotide sequence of primer FW secTEV_T180A
TCGGAAATACTAACAATTACTTCGCCTCTGTGCCCAAAAAC
SEQ ID Nr.: 25 Nucleotide sequence of primer FW pro-TEVcs-BDNF (MRV→ENL)
gggtcagagtggcgccggagattctcggacatgtttgcagcatct
SEQ ID Nr.: 26 Nucleotide sequence of primer RV pro-TEVcs-BDNF (MRV→ENL)
agatgctgcaaacatgtccgagaatctccggcgccactctgaccc
SEQ ID Nr.: 27 Nucleotide sequence of primer FW pro-TEVcs-BDNF (RRH→YFQ)
ccctcggcgggcagggtcagactggaaatagagattctcggacatgtttgc
SEQ ID Nr.: 28 Nucleotide sequence of primer RV pro-TEVcs-BDNF (RRH→YFQ)
gcaaacatgtccgagaatctctatttccagtctgaccctgcccgccgaggg

Primers Used for Gibson Assembly Reaction

SEQ ID Nr.: 29 Nucleotide sequence of primer FW Inserto_TEV-UniRap
CAACTTCCAAACTAAGAGCGGTGGATCAACCTGCGTGG
SEQ ID Nr.: 30 Nucleotide sequence of primer RV Inserto_TEV-UniRap
TGACACCATGCTAGACATTGATCCACCTTCCAGTTTTAGAAGCT
SEQ ID Nr.: 31 Nucleotide sequence of primer FW Open_TEV120/121
ATGTCTAGCATGGTGTCAGACACTAG
SEQ ID N.: 32 Nucleotide sequence of primer RV Open_TEV120/121
GCTCTTAGTTTGGAAGTTGGTTGT
SEQ ID Nr.: 33 Nucleotide sequence of primer FW Inserto_SecTEV-UniRap
CCAACTTCCAGACCAAATCCGGTGGATCAACCTGCGTGGTGCACTACACCGG
SEQ ID Nr.: 34 Nucleotide sequence of primer RV Inserto_SecTEV-UniRap
TGACACCATGCTAGACATTGATCCACCTTCCAGTTTTAGAAGCT
SEQ ID Nr.: 35 Nucleotide sequence of primer FW Open_SecTEV120/121
ATGAGCTCCATGGTCTCCGACAC
SEQ ID Nr.: 36 Nucleotide sequence of primer RV Open_SecTEV120/121
GGATTTGGTCTGGAAGTTGGTAG

1. A protein with inducible proteolytic activity comprising the polypeptide sequence of a protease and the polypeptide sequence of an activator-binding domain, wherein said domain in the absence of said activator inhibits the proteolytic activity of said protease and in the presence of said activator restores the proteolytic activity of said protease, wherein said activator is rapamycin or an analogue thereof.

2. The protein according to claim 1, wherein said domain is a domain constituted by the fusion of the FRB and FKBP domain.

3. The protein according to claim 1, wherein said domain has the polypeptide sequence SEQ ID NO: 1.

4. The protein according to claim 1, wherein said protease belongs to the C4 peptidase family.

5. The protein according to claim 1, wherein said protease is TEV protease having SEQ ID NO: 3 or SEQ ID NO: 4.

6. The protein according to claim 1, wherein said domain is inserted between A and B domains of said protease, wherein said A and B domains correspond to two inactive fragments of said protease.

7. The protein according to claim 1, wherein said protein comprises one or more linker sequences for binding between said protease and said domain, wherein said linkers have one of the following sequences: Gly-Gly-Ser-Gly-Gly-Gly (SEQ ID NO: 2), Gly-Gly-Ser and Gly.

8. The protein according to claim 1, wherein said protease is TEV protease having SEQ ID NO: 3 SEQ ID NO: 4. and wherein said domain is inserted between 5120 and M121 residues of said protease.

9. The protein according to claim 1, wherein said protease is TEV protease having SEQ ID NO: 3 or SEQ ID NO: 4, wherein one or more of the following mutations N23Q, T173G, C130S, I138T, S153N and T180A are inserted in the sequence of said protease.

10. The protein according to claim 1, wherein said protein with inducible proteolytic activity has SEQ ID NO: 5 (unica-TEV).

11. The protein according to claim 1, wherein said protein with inducible proteolytic activity has SEQ ID NO: 6 (unica-sec-TEV).

12. A nucleotide sequence codifying a protein with inducible proteolytic activity, wherein said protein has SEQ ID NO: 5 (unica-TEV) or SEQ ID NO: 6 (unica-sec-TEV).

13. A method for controlling maturation of a protein subject to proteolytic cleavage in a cell comprising contacting the cell with the protein of claim 1.

14. The method according to claim 13 for control in the cytosol or in other secretory pathways.

15. The method according to claim 13, wherein said protein subject to proteolytic cleavage is BDNF.

16. A method of purifying recombinant proteins of interest comprising contacting the proteins of interest with the protein according to claim 1.

17. A method for controlling the maturation in a cell of a protein subject to proteolytic cleavage comprising the following steps of:
a) co-expressing in a cell a protein with inducible proteolytic activity according to claim 1 and a protein subject to proteolytic cleavage
b) incubating said cell with the activators of said protein with proteolytic activity in order to induce the maturation of said protein.

18. The method according to claim 17, wherein the maturation of BDNF protein is induced.

19. A method of treating a disease associated with uncontrolled maturation of a disease-associated protein, comprising administering a therapeutically effective amount of the protein of claim 1 to a subject in need thereof.

20. A method of treating a disease associated with uncontrolled maturation of a disease-associated protein, comprising administering a therapeutically effective amount of the nucleotide sequence of claim 12 to a subject in need thereof


SEQUENCE LISTING (36 sequences)

SEQ ID NO: 1 - protein, 198 aa, Artificial sequence
  Synthetic construct
  Peptide sequence of activator-binding domain (1)..(198)
TCVVHYTGMLEDGKKFDSSRDRNKPFKFMLGKQEVIRGWEEGVAQMSVGQRAKLTISPDYAYGATGHGSGSGSGVKDLLQAWDLYYHVFRRISGPPGPGSGLWHEMWHEGLEEASRLYFGERNVKGMFEVLEPLHAMMERGPQTLKETSFNQAYGRDLMEAQEWCRKYMKSGSSGGSGSGIIPPHATLVFDVELLKLE

SEQ ID NO: 2 - protein, 6 aa, Artificial Sequence
  Synthetic construct
  Peptide sequence of a linker molecule (1)..(6)
GGSGGG

SEQ ID NO: 3 - protein, 236 aa, Artificial Sequence
  Synthetic construct
  Peptide sequence of wild-type TEV protease (1)..(236)
GESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMN

SEQ ID NO: 4 - protein, 261 aa, Artificial Sequence
  Synthetic construct
  Peptide sequence of sec-TEV protease (1)..(261)
MGWSLILLFLVAVATGVHSQGESLFKGPRDYNPISSTICHLTQESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSSTFPSSDGIFWKHWIQTKDGQCGSPLVSTRDGFIVGIHSASNFGNTNNYFTSVPKNFMELLTNQEAQQWVSGWRLNADSVLWGGHKVFMSKPEEPFQPVKEATQLMNEGGLE

SEQ ID NO: 5 - protein, 440 aa, Artificial Sequence
  Synthetic construct
  Peptide sequence of unica-TEV engineered protease (1)..(440)
GESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSGGSTCVVHYTGMLEDGKKFDSSRDRNKPFKFMLGKQEVIRGWEEGVAQMSVGQRAKLTISPDYAYGATGHGSGSGSGVKDLLQAWDLYYHVFRRISGPPGPGSGLWHEMWHEGLEEASRLYFGERNVKGMFEVLEPLHAMMERGPQTLKETSFNQAYGRDLMEAQEWCRKYMKSGSSGGSGSGIIPPHATLVFDVELLKLEGGSMSSMVSDTSCTFPSSDGTFWKHWIQTKDGQCGNPLVSTRDGFIVGIHSASNFTNTNNYFASVPKNFMELLTNQEAQQWVSGWRLNADSVLWGGHKVFMVKPEEPFQPVKEATQLMN

SEQ ID NO: 6 - protein, 468 aa, Artificial Sequence
  Synthetic construct
  Peptide sequence of unica-sec-TEV engineered protease (1)..(468)
MGWSLILLFLVAVATGVHSQGAQGESLFKGPRDYNPISSTICHLTQESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSGGSTCVVHYTGMLEDGKKFDSSRDRNKPFKFMLGKQEVIRGWEEGVAQMSVGQRAKLTISPDYAYGATGHGSGSGSGVKDLLQAWDLYYHVFRRISGPPGPGSGLWHEMWHEGLEEASRLYFGERNVKGMFEVLEPLHAMMERGPQTLKETSFNQAYGRDLMEAQEWCRKYMKSGSSGGSGSGIIPPHATLVFDVELLKLEGGSMSSMVSDTSSTFPSSDGTFWKHWIQTKDGQCGNPLVSTRDGFIVGIHSASNFGNTNNYFASVPKNFMELLTNQEAQQWVSGWRLNADSVLWGGHKVFMSKPEEPFQPVKEATQLMNEGGLE

SEQ ID NO: 7 - DNA, 711 bp, Artificial Sequence
  Synthetic construct
  Nucleotide sequence of wild-type TEV protease (1)..(711)
atgggagaaagcttgtttaaggggccgcgtgattacaacccgatatcgagcaccatttgtcatttgacgaatgaatctgatgggcacacaacatcgttgtatggtattggatttggtcccttcatcattacaaacaagcacttgtttagaagaaataatggaacactgttggtccaatcactacatggtgtattcaaggtcaagaacaccacgactttgcaacaacacctcattgatgggagggacatgataattattcgcatgcctaaggatttcccaccatttcctcaaaagctgaaatttagagagccacaaagggaagagcgcatatgtcttgtgacaaccaacttccaaactaagagcatgtctagcatggtgtcagacactagttgcacattcccttcatctgatggtatattctggaagcattggattcaaaccaaggatgggcagtgtggcagtccattagtatcaactagagatgggttcattgttggtatacactcagcatcgaatttcaccaacacaaacaattatttcacaagcgtgccgaaaaacttcatggaattgttgacaaatcaggaggcgcagcagtgggttagtggttggcgattaaacgctgactcagtattgtgggggggccataaagttttcatggtgaaacctgaagaaccttttcagccagttaaggaagcgactcaactcatgaat

SEQ ID NO: 8 - DNA, 786 bp, Artificial Sequence
  Synthetic construct
  Nucleotide sequence of sec-TEV protease (1)..(786)
atgggctggagcctgatcctcctgttcctcgtcgctgtggctacaggtgtgcactctcagatgggggaaagcctgttcaagggaccaagggactacaatccaatctcctcaactatctgccacctgactcaggaaagcgacggacataccacatctctgtacggaattggcttcgggcccttcatcattactaacaagcacctgtttcggagaaacaatggcaccctgctggtgcagagtctgcacggggtgttcaaggtcaaaaatactaccacactgcagcagcatctgattgacggacgagatatgatcattatccggatgccaaaggacttccccccttttccccagaagctgaagttccgggagccccagagggaggaacgcatctgcctggtgactaccaacttccagaccaaatccatgagctccatggtctccgacacctcttctacattcccttctagtgatggcatcttctggaagcactggatccagacaaaagacggacagtgcggcagtccactggtgtcaaccagagatgggtttattgtcggaatccattcagccagcaacttcggaaatactaacaattacttcacctctgtgcccaaaaacttcatggagctgctgactaatcaggaagcacagcagtgggtgagcggatggcgcctgaatgctgattccgtgctgtggggcgggcataaggtcttcatgagcaaacctgaagagccatttcagcccgtcaaggaagccacccagctgatgaacgaagggggcctggaa

SEQ ID NO: 9 - DNA, 1323 bp, Artificial Sequence
  Synthetic construct
  Nucleotide sequence of unica-TEV engineered protease (1)..(1323)
atgggagaaagcttgtttaaggggccgcgtgattacaacccgatatcgagcaccatttgtcatttgacgaatgaatctgatgggcacacaacatcgttgtatggtattggatttggtcccttcatcattacaaacaagcacttgtttagaagaaataatggaacactgttggtccaatcactacatggtgtattcaaggtcaagaacaccacgactttgcaacaacacctcattgatgggagggacatgataattattcgcatgcctaaggatttcccaccatttcctcaaaagctgaaatttagagagccacaaagggaagagcgcatatgtcttgtgacaaccaacttccaaactaagagcggtggatcaacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacggttcgggctccggatcaggcgtcaaggacctcctccaagcctgggacctctattatcatgtgttccgacgaatctcaggtcctccaggacctggatcaggtctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggtcatcagggggctccggatcaggcatcatcccaccacatgccactctcgtcttcgatgtggagcttctaaaactggaaggtggatcaatgtctagcatggtgtcagacactagttgcacattcccttcatctgatggtacgttctggaagcattggattcaaaccaaggatgggcagtgtggcaatccattagtatcaactagagatgggttcattgttggtatacactcagcatcgaatttcaccaacacaaacaattatttcgcaagcgtgccgaaaaacttcatggaattgttgacaaatcaggaggcgcagcagtgggttagtggttggcgattaaacgctgactcagtattgtgggggggccataaagttttcatggtgaaacctgaagaaccttttcagccagttaaggaagcgactcaactcatgaat

SEQ ID NO: 10 - DNA, 1404 bp, Artificial Sequence
  Synthetic construct
  Nucleotide sequence of unica-sec-TEV engineered protease (1)..(1404)
atgggctggagcctgatcctcctgttcctcgtcgctgtggctacaggtgtgcactctcagggcgcgcaaggggaaagcctgttcaagggaccaagggactacaatccaatctcctcaactatctgccacctgactcaggaaagcgacggacataccacatctctgtacggaattggcttcgggcccttcatcattactaacaagcacctgtttcggagaaacaatggcaccctgctggtgcagagtctgcacggggtgttcaaggtcaaaaatactaccacactgcagcagcatctgattgacggacgagatatgatcattatccggatgccaaaggacttccccccttttccccagaagctgaagttccgggagccccagagggaggaacgcatctgcctggtgactaccaacttccagaccaaatccggtggatcaacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacggttcgggctccggatcaggcgtcaaggacctcctccaagcctgggacctctattatcatgtgttccgacgaatctcaggtcctccaggacctggatcaggtctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggtcatcagggggctccggatcaggcatcatcccaccacatgccactctcgtcttcgatgtggagcttctaaaactggaaggtggatcaatgagctccatggtctccgacacctcttctacattcccttctagtgatggcaccttctggaagcactggatccagacaaaagacggacagtgcggcaatccactggtgtcaaccagagatgggtttattgtcggaatccattcagccagcaacttcggaaatactaacaattacttcgcctctgtgcccaaaaacttcatggagctgctgactaatcaggaagcacagcagtgggtgagcggatggcgcctgaatgctgattccgtgctgtggggcgggcataaggtcttcatgagcaaacctgaagagccatttcagcccgtcaaggaagccacccagctgatgaacgaagggggcctggaa

SEQ ID NO: 11 - protein, 19 aa, Artificial Sequence
  Synthetic construct
  Peptide sequence of the signal leader peptide (1)..(19)
MGWSLILLFLVAVATGVHS

SEQ ID NO: 12 - protein, 6 aa, Artificial Sequence
  Synthetic construct
  Cleavage peptide sequence of TEV protease (1)..(6)
ENLYFQ

SEQ ID NO: 13 - DNA, 43 bp, Artificial Sequence
  Synthetic construct
  Nucleotide sequence of primer RV TEV_I138T (1)..(43)
ccaatgcttccagaacgtaccatcagatgaagggaatgtgcaa

SEQ ID NO: 14 - DNA, 43 bp, Artificial Sequence
  Synthetic construct
  Nucleotide sequence of primer FW TEV_I138T (1)..(43)
ttgcacattcccttcatctgatggtacgttctggaagcattgg

SEQ ID NO: 15 - DNA, 35 bp, Artificial Sequence
  Synthetic construct
  Nucleotide sequence of primer RV TEV_S153N (1)..(35)
ttgatactaatggattgccacactgcccatccttg

SEQ ID NO: 16 - DNA, 35 bp, Artificial Sequence
  Synthetic construct
  Nucleotide sequence of primer FW TEV_S153N (1)..(35)
caaggatgggcagtgtggcaatccattagtatcaa

SEQ ID NO: 17 - DNA, 42 bp, Artificial Sequence
  Synthetic construct
  Nucleotide sequence of primer RV TEV_T180A (1)..(42)
gtttttcggcacgcttgcgaaataattgtttgtgttggtgaa

SEQ ID NO: 18 - DNA, 42 bp, Artificial Sequence
  Synthetic construct
  Nucleotide sequence of primer FW TEV_T180A (1)..(42)
ttcaccaacacaaacaattatttcgcaagcgtgccgaaaaac

SEQ ID NO: 19 - DNA, 33 bp, Artificial Sequence
  Synthetic construct
  Nucleotide sequence of primer RV secTEV_I138T (1)..(33)
ccagtgcttccagaaggtgccatcactagaagg

SEQ ID NO: 20 - DNA, 33 bp, Artificial Sequence
  Synthetic construct
  Nucleotide sequence of primer FW secTEV_I138T (1)..(33)
ccttctagtgatggcaccttctggaagcactgg

SEQ ID NO: 21 - DNA, 29 bp, Artificial Sequence
  Synthetic construct
  Nucleotide sequence of primer RV secTEV_S153N (1)..(29)
gacaccagtggattgccgcactgtccgtc

SEQ ID NO: 22 - DNA, 29 bp, Artificial Sequence
  Synthetic construct
  Nucleotide sequence of primer FW secTEV_S153N (1)..(29)
gacggacagtgcggcaatccactggtgtc

SEQ ID NO: 23 - DNA, 41 bp, Artificial Sequence
  Synthetic construct
  Nucleotide sequence of primer RV secTEV_T180A (1)..(41)
gtttttgggcacagaggcgaagtaattgttagtatttccga

SEQ ID NO: 24 - DNA, 41 bp, Artificial Sequence
  Synthetic construct
  Nucleotide sequence of primer FW secTEV_T180A (1)..(41)
tcggaaatactaacaattacttcgcctctgtgcccaaaaac

SEQ ID NO: 25 - DNA, 45 bp, Artificial Sequence
  Synthetic construct
  Nucleotide sequence of primer FW pro-TEVcs-BDNF (MRV′ENL) (1)..(45)
gggtcagagtggcgccggagattctcggacatgtttgcagcatct

SEQ ID NO: 26 - DNA, 45 bp, Artificial Sequence
  Synthetic construct
  Nucleotide sequence of primer RV pro-TEVcs-BDNF (MRV′ENL) (1)..(45)
agatgctgcaaacatgtccgagaatctccggcgccactctgaccc

SEQ ID NO: 27 - DNA, 51 bp, Artificial Sequence
  Synthetic construct
  Nucleotide sequence of primer FW pro-TEVcs-BDNF (RRH′YFQ) (1)..(51)
ccctcggcgggcagggtcagactggaaatagagattctcggacatgtttgc

SEQ ID NO: 28 - DNA, 51 bp, Artificial Sequence
  Synthetic construct
  Nucleotide sequence of primer RV pro-TEVcs-BDNF (RRH′YFQ) (1)..(51)
gcaaacatgtccgagaatctctatttccagtctgaccctgcccgccgaggg

SEQ ID NO: 29 - DNA, 38 bp, Artificial Sequence
  Synthetic construct
  Nucleotide sequence of primer FW Inserto_TEV-UniRap (1)..(38)
caacttccaaactaagagcggtggatcaacctgcgtgg

SEQ ID NO: 30 - DNA, 44 bp, Artificial Sequence
  Synthetic construct
  Nucleotide sequence of primer RV Inserto_TEV-UniRap (1)..(44)
tgacaccatgctagacattgatccaccttccagttttagaagct

SEQ ID NO: 31 - DNA, 26 bp, Artificial Sequence
  Synthetic construct
  Nucleotide sequence of primer FW Open_TEV120/121 (1)..(26)
atgtctagcatggtgtcagacactag

SEQ ID NO: 32 - DNA, 24 bp, Artificial Sequence
  Synthetic construct
  Nucleotide sequence of primer RV Open_TEV120/121 (1)..(24)
gctcttagtttggaagttggttgt

SEQ ID NO: 33 - DNA, 52 bp, Artificial Sequence
  Synthetic construct
  Nucleotide sequence of primer FW Inserto_SecTEV-UniRap (1)..(52)
ccaacttccagaccaaatccggtggatcaacctgcgtggtgcactacaccgg

SEQ ID NO: 34 - DNA, 44 bp, Artificial Sequence; identical to SEQ ID NO: 30
  Synthetic construct
  Nucleotide sequence of primer RV Inserto_SecTEV-UniRap (1)..(44)

SEQ ID NO: 35 - DNA, 23 bp, Artificial Sequence
  Synthetic construct
  Nucleotide sequence of primer FW Open_SecTEV120/121 (1)..(23)
atgagctccatggtctccgacac

SEQ ID NO: 36 - DNA, 23 bp, Artificial Sequence
  Synthetic construct
  Nucleotide sequence of primer RV Open_SecTEV120/121 (1)..(23)
ggatttggtctggaagttggtag